(19) United States
(12) Patent Application Publication
(10) Pub. No.: US 2016/0047819 A1
(43) Pub. Date: Feb. 18, 2016

(54) METHOD FOR DIAGNOSING VAGINAL INFECTIONS

(71) Applicant: ALFA WASSERMANN S.p.A., Alanno (IT)

(72) Inventors: Giuseppe Claudio VISCOMI, Bologna (IT); Fiorella CALANNI, Bologna (IT); Beatrice VITALI, Bologna (IT); Federica CRUCIANI, Bologna (IT)

(21) Appl. No.: 14/776,716

(22) PCT Filed: Mar. 4, 2014
(86) PCT No.: PCT/IB2014/059427

§ 371 Date: Sep. 14, 2015

Related U.S. Application Data
(60) Provisional application No. 61/794,385, filed on Mar. 15, 2013.

(51) Int. Cl. G01N 33/68

(52) U.S. Cl. CPC G01N 33/6848; G01N 2800/26; G01N 2800/52; G01N 2800/56; G01N 2570/00; G01N 2560/00

(57) ABSTRACT

Diagnostic methods for evaluating vaginal infections comprising the use of specific proteins are described. Also described here is the use of specific proteins in a diagnostic method for evaluating recovery from the infections following antibiotic treatment of vaginal infections and predicting the recovery and remission of the infection. Diagnostic methods involving the use of specific proteins for evaluating recovery from the vaginal infections following rifaximin treatment and predicting the recovery and remission of the infection are also described.

### FIELD OF THE INVENTION

The present invention relates to diagnostic methods for evaluating vaginal infections comprising the use of specific proteins. The invention further relates to the use of specific proteins in a diagnostic method for evaluating recovery from the infections following antibiotic treatment of vaginal infections and predicting the recovery and remission of the infection. The invention also relates to diagnostic methods involving the use of specific proteins for evaluating recovery from the vaginal infections following rifaximin treatment and predicting the recovery and remission of the infection

### BACKGROUND

Rifaximin (INN, see The Merck Index, XIII ed., 8304, CAS No. 80621-81-4), IUPAC nomenclature 2S,16Z,18E,20S,21S,22R,23R,24R,25S,26S,27S,28E)-5,6,21,23,25 pentahydroxy-27-methoxy-2,4,11,16,20,22,24,26-octamethyl-2,7-(epoxypentadeca(1,11,13)trienimine)benzofuro(4,5-e)pyrido(1,2-a benzimidazole-1,15(2H)dione, 25-acetate) is a semi-synthetic antibiotic drug belonging to the rifampicin group, more precisely a pyrido-imidazo-rifamycin, as described in IT 1154655. EP 0 161 534 describes a production process starting from Rifamycin O (The Merck Index XIII ed., 8301).

U.S. Pat. No. 7,045,620, EP 1557421B1, EP 1676847B1, EP 1676848B1, WO2005/044823, WO2006/094662 describe crystalline forms α, β, γ, δ and ε of rifaximin each of which are incorporated by reference in their entirety. WO2008/155728 and US 2009/312357 describe processes for obtaining amorphous forms each of which are incorporated by reference in their entirety. WO2009/108730 describes polymorphous forms of rifaximin named zeta, eta, α-dry, iota, β-1, β-2 and ε-dry each of which are incorporated by reference in their entirety. WO2011/153444 describes polymorphous forms κ and θ and WO 2011/156897 describes polymorphous forms named APO-1 and APO-2 each of which are incorporated by reference in their entirety. Viscomi G. et al., Cryst. Eng Comm., 2008, 10 1074-1081(2008) describes polymorphous α, β, γ, δ, ε, the process for obtaining them and their chemical-physical and biological properties which is incorporated by reference in their entirety.

Rifaximin is an antibiotic drug active against Gram-positive and Gram-negative bacteria, characterized by a low systemic absorption, negligible when administered via the oral route, as described by Descombe J. J. et al., Int. J. Clin. Pharmacol. Res., 14 (2), 51-56, (1994); it is known for its antibacterial activity, exerted, for instance, against bacteria localized in the gastrointestinal tract causing intestinal infections, diarrhea and irritable bowel syndrome (IBS), bacterial growth in the small intestine or “small intestinal bacterial overgrowth” (SIBO), which is also known to be associated with Crohn's disease (CD), pancreatic insufficiency, enteritis, fibromyalgia. Rifaximin plays a relevant role in the therapy of infectious and inflammatory bowel diseases, both in the acute and in the chronic phase.

The different forms of rifaximin are associated to different levels of systemic absorption. Rifaximin is presently authorized for the treatment of acute and chronic pathologies whose etiology is partially or completely related to Gram-positive and Gram-negative intestinal bacteria, such as diarrheic syndromes caused by an altered balance of the intestinal microbial flora such as summer diarrheas, traveler's diarrhea and enterocolitis. Rifaximin is useful in the pre- and post-surgical prophylaxis of infectious complications following gastroenteric tract surgery, as an adjuvant in hyperammonaemias therapy and in the reduction of the risk of acute episodes of hepatic encephalopathy.

Rifaximin can also be useful in treating “restless-legs syndrome”; for the prevention of spontaneous bacterial peritonitis in patients affected by hepatic insufficiency and in the infections induced by the chronic use of proton pump inhibitors.

Furthermore, the fact that rifaximin is poorly absorbed systemically is advantageous for the aforesaid applications, since rifaximin is not toxic, even at high doses and reduces the incidence of undesired side effects such as, for instance, the selection of antibiotic-resistant bacterial strains and the risk of possible pharmacological interactions.

Rifaximin's characteristics make it a compound useful in topical treatments, such as treatments of vaginal infections, for example bacterial vaginosis.

Vaginal infection is a frequent pathology among women and childbearing age and a percentage of 40-50% is represented by bacterial vaginosis. When it is symptomatic and without complications, bacterial vaginosis is characterized by malodorous vaginal discharges, is not associated with an inflammatory clinical picture (vaginosis), and is attributed to an alteration of the vaginal ecosystem.

Bacterial vaginosis is characterize by an imbalance in the ecology of the normal microbiota wherein the depletion of lactobacilli and proliferation of anaerobic bacteria occur.

The normal vaginal flora of a healthy woman, due to the prevailing presence of Lactobacilli, in particular Lactobacillus crispatus and gasseri, produces hydrogen peroxide and maintains an acid vaginal pH, thus inhibiting the growth of most pathogenic microorganisms.

In bacterial vaginosis, Lactobacillus bacteria are replaced by an excessive growth, even a thousand times higher than normal values, of facultative anaerobic and aerobic bacteria, mainly represented by Gardnerella vaginalis, which is present in nearly all women affected by bacterial vaginosis, by Mycoplasma hominis, by Gram-negative anaerobic bacteria such as Bacteroides and Prevotella, by anaerobes such as Peptostreptococcus, by Gram-positive anaerobes such as Mobiluncus, which is present in 50% of the cases, and by Gram-positive bacilli such as Atopobium vaginale, which is present in 95% of cases of bacterial vaginosis.

Factors predisposing women to the onset of bacterial vaginosis include being of childbearing age, race, socioeconomic status, frequent use of vaginal lavage, smoking and sexual activity with multiple partners. On the other hand, taking estroprogestinic drugs seems to play a protective role. Also, a hormonal component was found to be involved in the aetiopathogenesis of bacterial vaginosis, since this pathology is mainly found in fertile-aged women.

Bacterial vaginosis can be related to several serious gynecological and obstetrical complications, such as, for instance: pelvic inflammatory disease, frequent cause of sterility and ectopic pregnancy; infection of surgical injury after gynecologic surgery; premature rupture of the membranes in pregnant women; premature labor and abortion. Although it is not considered a sexually transmitted disease, bacterial vaginosis is associated to an increased risk of catching sexually transmitted pandemic diseases, including the HIV virus infection, both for non-pregnant and pregnant women. In the latter, it also determines an increased risk of transmission of HIV virus from the mother to the fetus.

The etiology of bacterial vaginosis is not completely understood; however, treatments aim to induce both a clinical and a microbiological recovery and, when possible, to avoid the relapse of infection. Therefore, an ideal therapy should be effective at reducing pathogenic species and at the same time, it should also encourage the restoration and proliferation of Lactobacillus protective species with the aim of preventing possible disease relapses.

The guidelines of the Centers for Disease Control (CDC), 2010, 59, NoRR-12 state that all women affected by bacterial vaginosis, who are symptomatic and non-pregnant, should be treated with antibiotic therapy. In this regard, the CDC suggests, as first therapeutic approach, antibiotic treatments such as, for instance: metronidazole, oral tablets 500 mg, twice a day for 7 days; or metronidazole, vaginal gel, 0.75%, an applicator (5 g once a day for 5 days or clindamycin, vaginal cream, 2%, an applicator (5 g) once a day for 7 days. Both metronidazole and clindamycin, administered either via the systemic route (orally) or via local route (vaginally), are effective in treating bacterial vaginosis. However, the inhibitory action of both of these drugs against Lactobacillus protective flora limits their efficacy in preventing relapses, as described by Simoes J A et al., Infect. Dis. Obstet. Gynecol. 2001, 9(1), 41-45.

Furthermore, both of the above mentioned antibiotics are associated with systemic side effects, some of them particularly relevant, such as, for instance, neurological reactions for metronidazole or pseudomembranose colitis for clindamycin, even when administered via vaginal route. Moreover, if repeatedly administered, both metronidazole and clindamycin can induce microbiological resistances not only at the vaginal level, but also at the systemic level, since they are systemically absorbed even after vaginal administration.

EP 0547294 describes compositions containing rifaximin in amounts between 50 and 500 mg which are stated to be useful in treating vaginal infections caused by microorganisms susceptible to rifaximin. In particular, EP 0547294 describes a clinical trial carried out with a preparation of rifaximin vaginal foam and cream, containing 200 mg rifaximin and describes compositions for treating bacterial vaginosis containing rifaximin in capsules, ovules and tablets. Table 1 of EP 0547294 describes that rifaximin exerts an important antibacterial activity both against pathogenic bacteria such as Gardnerella vaginalis, Bacteroides bivious-disiens, Mobiluncus and also against non-pathogenic bacteria such as Lactobacilli, which are commonly present in vaginal discharge.

The inhibition of Lactobacilli, whose presence is beneficial for maintaining the healthy vaginal environment, must be considered a detrimental event with regard to therapeutic efficacy. In fact, as already stated, the acid environment generated by lactobacilli is an essential condition for preventing pathogenic bacteria colonization.

Table 1 of EP 0547292 also shows that rifaximin inhibitory action (MIC50 and MIC90) against Lactobacilli is equal to, or even higher than, its action against pathogenic bacteria, such as, for instance, Gardnerella vaginalis, Mobiluncus spp, Bacteroides bivius-disiens. Thus, when administered via the vaginal route, rifaximin indiscriminately acts on the whole bacterial flora, including Lactobacilli.

Debbia A. et al., J Chemother 20, (2), 186-194, 2008, reports that rifaximin exhibits a time-dependent bacterial activity.

U.S. Ser. No. 13/559,613 describes rifaximin pharmaceutical compositions effective in treating vaginal infections, providing for an appropriate period of time of exposure to rifaximin and local concentrations of rifaximin useful in treating vaginal infections, which do not reduce the Lactobacilli concentration, which is important for the prevention of relapse of vaginal infections. Moreover, U.S. Ser. No. 13/559,013 describes clinical study wherein rifaximin is efficacious in the treatment of vaginal infections at daily dosage less than 100 mg/day

The diagnosis of bacterial vaginosis can be based upon clinical and/or microbiological criteria. The clinical diagnosis is carried out according to Amsel clinical criteria, as described by Amsel R. et al. in Am J Med 1983; 74(1): 14-22. The diagnosis is positive when at least three out of the four following symptoms are reported: 1) vaginal discharges which are homogeneous and adhering to the vaginal walls; 2) whiff test positivity (development of “fishy odor” after the addition of 10% potassium hydroxide to vaginal discharge); 3) vaginal pH higher than 4.5, and 4) an amount greater than 20% of clue cells (squamous epithelium vaginal cells coated with bacteria, identified by fresh microscopic examination).

The microbiological diagnosis is based on the calculation of the Nugent score, which includes microscopic examination of vaginal discharges by means of Gram staining. The presence and the quantity of three different vaginal bacterial species is determined. In particular, a low score is obtained if the Lactobacilli concentration is high, the score increases if the presence of Gardnerella and Bacteroidi is ascertained, and the score is even higher if also the presence of Mobiluncus is ascertained. A resulting score between 0 and 3 is representative of vaginal flora of a healthy woman, a score between 4 and 6 indicates that vaginal flora is starting to be altered, and a score between 7 and 10 indicates a certain diagnosis of bacterial vaginosis, as described by Nugent R P et al., J Clin Microbiol 1991, 29(2), 297-301.

In recent years further diagnostic molecular techniques have been developed, such as PCR-DGGE and real-time PCR, based upon the sequence analysis of DNA and allowing the identification of a microbial composition of the vaginal ecosystem, as described by Zhou X et al. in Microbiology 2004, 150 (Pt8), 2565-2573 and in Appl Environ Microbiol 2004, 70(6), 3575-3581. The polymerase chain reaction (PCR) amplifies a single or a few copies of DNA across several orders of magnitude, generating thousands to millions of copies of a particular DNA sequence, and is useful for identifying a gene or genes which are below the level of detection using other methods.

### SUMMARY OF THE INVENTION

This invention relates to a new method for diagnosing vaginal infections, in particular bacterial vaginosis (BV). The diagnostic method of this invention is minimally invasive and allows the evaluation of BV by the use of specific proteins, e.g., by determining the number and types of proteins in vaginal fluid of a patient compared with those of a reference sample of vaginal fluid that represents a healthy or non-infected state. One embodiment of the present invention is a method for diagnosing vaginal infections by means of characterizing specific proteins present in the vaginal fluid. One embodiment relates to the use of the characterized proteins for selecting the most efficacious antibiotic and dosage to obtain remission from BV or to eradicate BV in the patient.

In one embodiment, the invention provides a method of diagnosing a vaginal bacterial infection comprising subjecting a vaginal fluid sample to proteomic analysis; determining the proteins having altered levels of expression in the test fluid sample compared with the levels of expression of the proteins in a reference sample wherein a decrease or increase in expression levels of one or more proteins diagnose the vaginal infection. In particular embodiments, the one or more proteins are selected from those listed in Tables 1 and 2. In some embodiments, the expression increase between the test sample and reference sample is a ratio in the range from about 1.5 to about 40. In other embodiments, the protein expression decrease between the test sample and reference sample is a ratio in the range from about −1.5 to about −5650.

In another embodiment the status of remission and recovery may be assessed by comparing the levels of expression of proteins in a sample from an individual who had responded to treatment following infection with the level of protein in a reference sample representative of a healthy individual.

In another embodiment, the invention provides a method of diagnosing the status of remission from a bacterial vaginal infection of an individual undergoing testing for remission after antibiotic treatment, by subjecting a vaginal fluid sample obtained from the individual undergoing testing after antibiotic treatment to proteomic analysis; and determining the proteins having altered levels of expression in the test fluid sample compared with the levels of expression of the proteins in a reference sample representing fluid from a BV infected individual (preferably, the same individual before treatment), wherein a decrease or increase in expression levels of at least one protein in the test versus the reference sample diagnoses the status of remission from BV after antibiotic treatment.

In a further embodiment, the invention provides a method of diagnosis for predicting remission and recovery of BV based on proteomic analysis of vaginal sample of infected women, wherein the BV is identified when the levels of expressed proteins is in a ratio greater than 1 in comparison with those of healthy or uninfected women. In some embodiments, a method of selecting an optimal or efficacious dose of antibiotic and time of treatment is provided based on the decrease or the increase in protein expression levels in the test sample versus the reference sample. Efficacious treatment can also be identified comparing the change in protein expression before and after various treatments wherein the most efficacious treatment corresponds to the pool having the greatest number of differentially expressed proteins. Non responsive patients are identified as those who are not characterized as in remission after treatment by antibiotics, e.g. rifaximin.

In another embodiment, the invention provides a test kit for diagnosing a vaginal bacterial infection or evaluating remission or efficacy of treatment according to the methods disclosed herein is also provided. The kit includes at least one protein useful for identifying the vaginal infection, such as one identified in Tables 7 and 8, preferably one identified in Table 1 or 2, and instructions for carrying out the method of diagnosing vaginal infection using mass spectrometry.

In another embodiment, the invention provides an use of antibiotics for treating a vaginal bacterial infection in an individual comprising administering a pharmaceutical composition to the individual in therapeutically effective amounts based on a diagnosis of the infection comprising subjecting a vaginal fluid sample to proteomic analysis; determining the proteins having altered levels of expression in the test fluid sample compared with the levels of expression of the proteins in a reference sample wherein a decrease or increase in expression levels of one or more proteins diagnose the vaginal infection.

The specific proteins identified herein are useful i) to evaluate remission from a bacterial vaginal infection in an individual being tested, ii) to predict or determine at the time of diagnosis, the probability that the bacterial vaginal infection will go into remission by administering antibiotic treatment, and iii) to select or identify the most efficacious antibiotic and/or dosage for obtaining remission from the infection. In particular, the present invention describes the use of specific proteins for evaluating the remission of BV after the treatment with rifaximin. Moreover, it is possible to predict or determine the possibility that a patient undergoing testing will go into remission from the infection after antibiotic treatment.

In another embodiment the invention also provides a method for evaluating and predict the efficacy of the rifaximin treatment of women affected by BV.

In a particular embodiment the invention provide a diagnostic method for evaluating efficacy of rifaximin treatment during the treatment and before the treatment.

In another particular embodiment the invention provides a diagnostic method for predicting if the women affected by BV will be or will be not in remission, by the presence of specific proteins in vaginal fluid.

The present invention overcomes drawbacks and problems in the art by providing a method for diagnosing vaginal infections, evaluating the efficacy of methods of treating vaginal infections, and identifying non-responders to particular courses of treatment based on the comparison of proteomic profiles of vaginal fluid sampled at various times before, during and after a course of therapy for treating the vaginal infection.

In a another embodiment, a method for diagnosis of vaginal infections is provided, comprising comparing the proteomic profile of a test sample of a vaginal fluid with the proteomic profile of a normal or reference sample of a vaginal fluid and determining the presence of the vaginal infection if the total number of proteins of Table 1 or Table 2 is at least 1 or more.

In an another embodiment, a method for evaluating of the efficacy of treatment of vaginal infections is provided, comprising comparing the proteomic profiles of a test sample of a vaginal fluid during, or after, a course of therapy with the proteomic profiles of a sample of vaginal fluid taken before a course of therapy, or at an earlier point during the course of therapy, and determining the remission of the vaginal infection if the total number of proteins of Table 1 or Table 2 is at least 1

In another embodiment, a method for identifying the most efficacious treatment of vaginal infections is provided, comprising administering a distinct course of treatment to each pool of patients diagnosed with vaginal infection, comparing the proteomic profiles of test samples of vaginal fluid during or after a course of therapy and determining most efficacious treatment by identifying the pool of patients having a proteomic profile having the greatest number of differentially expressed proteins. Notably, the samples to be compared should be taken at the same time intervals so as to provide a meaningful comparison.

In another embodiment, a method for predicting remission and recovery during or following treatment of vaginal infections is provided, comprising comparing the proteomic profiles of a test sample of a vaginal fluid from a patient diagnosed with vaginal infection with the proteomic profiles of a normal or a reference sample of vaginal fluid, and predicting the remission of vaginal infection if the total number of is at least 1 or more 10, wherein the proteins are selected from Table 1 or Table 2 or from a combination of both tables.

In accordance with the described methods, the specific proteins presented in Table 1, Table 2, or a combination thereof are also termed “biomarkers”. These proteins present in vaginal fluid are selected by the Table 7 and 8 and they represent the most significant proteins in the vaginal fluid in women affected by vaginal infection in respect to health women. Certain preferred proteins also defined “biomarkers” include Vitamin D binding protein, Desmocollin-2, Calcium-activated chloride channel regulator 4, Catalase, Small proline-rich protein 3, Galectin-3-binding protein, Hemopexin, Immunoglobulin family, Intermediate filament family, Lipocalin family, Alpha 1-acid glycoprotein 1, Alpha-1-acid glycoprotein 2, Neutrophil gelatinase-associated lipocalin, Limphocyte-specific protein 1, Myeloblastin, Perilipin-3, Perilplakin, Protein S100-A9, Protein S100-A7, and Superoxide dismutase [Cu—Zn].

### BRIEF DESCRIPTION OF THE FIGURES

FIG. 1: Design of the collected sample of vaginal fluid

FIG. 2: Multivariate analysis of MS/MS data. Molecular analysis of vaginal microbiota composition.

### DETAILED DESCRIPTION OF THE EMBODIMENTS

Unless defined otherwise, technical and scientific terms used herein have the same meaning as commonly understood by one of ordinary skill in the art to which this invention pertains. Singleton et al., Dictionary of Microbiology and Molecular Biology 2nd ed., J. Wiley & Sons (New York, N.Y. 1994) provides one skilled in the art with a general guide to many of the terms used in the present application.

The term “proteome” is used herein to describe a significant portion of proteins in a biological sample at a given time. The concept of proteome is fundamentally different from the genome. The term “proteome” or “proteomic profile” is used to refer to a representation of the expression pattern, of a plurality of proteins in a biological sample, e.g., a vaginal fluid, at a given time. The proteomic profile can, for example, be represented as a mass spectrum, but other representations, e.g., chromatographic spectrums, based on any physicochemical or biochemical properties of the proteins, including a spectrum of identified or expressed proteins, or fragments thereof, are also included. Thus the proteomic profile may, for example, be based on differences in the electrophoretic properties of proteins, as determined by two-dimensional gel electrophoresis, e.g. by 2-D PAGE, and can be represented, e.g., as a plurality of spots in a two-dimensional electrophoresis gel. Alternatively, the proteomic profile may be based on differences in protein isoelectric point and hydrophobicity, as determined by two-dimensional liquid chromatography, and can be represented, e.g., as a computer generated virtual two-dimensional map or they may separated on the base of their molecular weight in a system, for example, based on a membrane having different porosity capable of separating proteins having different molecular weights.

The term proteins or “biomarkers” have particularly important diagnostic value. Proteins in the vaginal fluid can increase or decrease with the onset of, during the course of, and/or in the remission of a pathological condition, e.g., vaginal infection. The number of differentially expressed proteins or biomarkers has a particularly important diagnostic, evaluative, and predictive value. For example, in the present method of evaluating the efficacy of an antibiotic treatment, the greater the number of proteins that are differentially expressed, the stronger the indication that the treatment is effective. If there are too few proteins that are differentially expressed, a patient may be identified as a non-responder and a course of therapy will need to be adjusted in order to achieve remission of the disease for that patient, e.g., changing the antibiotic, changing the dosage, changing the dosing frequency. Also, the most efficacious treatment may be identified by comparing the number of differentially expressed proteins between different pools of patients treated by different therapies. The therapy resulting in the greatest number of differentially expressed proteins can be selected as the most efficacious.

Samples from different sources, such as healthy vaginal fluid (reference sample, normal or uninfected sample) and a vaginal fluid obtained from a patient diagnosed with bacterial vaginosis (test sample), can be compared to detect proteins that are up- or down-regulated (“biomarkers”). These proteins can be excised for identification and full characterization, e.g., using peptide-mass fingerprinting and/or mass spectrometry and sequencing methods, or the normal and/or disease-specific proteome map can be used directly for the diagnosis of the disease of interest (bacterial vaginosis), or to confirm the presence, absence or status of the disease. The vaginal fluid (VF, also referred to as cervical-vaginal fluid, CVF) is a complex biological fluid consisting of water, electrolytes, low molecular weight organic compounds (glucose, amino acids and lipids), and a vast array of proteins and proteolytic enzymes arising from plasma transudate, cervical/vaginal epithelial cells, endocervix, chorion and vaginal microbiota as described by Dasari S. et al in J Proteome Res 2007, 6, 1258-68 and Zegels et al n Proteome Sci 2010, 8, 63. Vaginal fluid forms the first line of defense against external pathogens, signals fertility, and aids insemination, pregnancy, and labor as described by Bigelow, Hum Reprod 2004, 19, 884-92.

Collection of vaginal fluid from a patient is minimally invasive and relatively safe, and therefore it is especially convenient and useful as a source of biomarkers for diagnosis of pathological conditions such as vaginal infections (e.g., BV) as well as for the development of treatment, diagnosis, and prevention strategies.

Diagnosis of a vaginal infection includes identifying a “patient response” which can be assessed using any endpoint indicating a change in status of the vaginal infection, including, without limitation, (1) inhibition, at least to some extent, of the progression of a vaginal infection, (2) prevention of the vaginal infection, (3) remission, at least to some extent, of one or more symptoms or indicators associated with the vaginal infection, such as Nugent Score or Amsel's criteria; and/or (4) cure wherein all symptoms or indicators associated with the pathologic condition are absent and/or the individual and vaginal fluid thereof are restored to a healthy condition at a given point of time following treatment.

The term “treatment” or “course of treatment” refers to both therapeutic treatment and prophylactic or preventative measures, wherein the object is to prevent, or slow down (cause remission) or recovery (eliminate all indicators of pathological condition) the targeted pathologic condition or disorder. Treatment encompasses the selection and administration of one or more pharmacologically active substances (drugs), the dosages and frequencies thereof as well as selection of the dosage form for best efficacy. Those in need of treatment include those already with the disorder (e.g. BV) as well as those prone to have the disorder (relapse infections) or those in whom the disorder is to be prevented.

In some embodiments of the inventive methods, treating a vaginal bacterial infection in an individual comprises administering a pharmaceutical composition to the individual in therapeutically effective amounts based on a diagnosis of the infection comprising subjecting a vaginal fluid sample to proteomic analysis. Forms of rifaximin and pharmaceutical compositions of rifaximin are described in U.S. Pat. Nos. 7,045,620; 8,158,781; 8,173,801; 7,902,206; 8,217,054; 7,923,553; 8,158,644; 8,193,196; and 6,140,355 which are all incorporated by reference in their entirety.

The present invention concerns methods and means for a non invasive diagnosis of a vaginal infection, based upon differential protein expression as determined by a comparison of the proteomic profile of vaginal fluid obtained from a patient, or different pools of patients, e.g., healthy patients, diseased patients and patients at various stages of treatment.

As identified by the described methods herein, the specific proteins and protein families present in the vaginal fluid that increase or decrease with the onset and/or remission of a vaginal infection are termed “biomarkers”. These biomarkers can be objectively measured and, according to the methods described herein, used to i) diagnose vaginal infections, ii) predict and/or evaluate the efficacy of treatment of a vaginal infection, and to iii) identify the most efficacious treatment such as antibiotic, dosage and frequency. These proteins can be determined by the use of analytical techniques for protein determination such as proteomic techniques known to those having skill in the art, e.g., mass spectroscopy.

Described herein are the use of specific proteins (biomarkers) present in the vaginal fluid for diagnosing and/or evaluating the state of bacterial vaginosis at various stages of the disease including remission and cure. The specific proteins are characterized by analyzing the proteome profiles of vaginal fluid using the techniques known in the field of proteomic analysis.

In one aspect, the present invention advantageously provides a new and minimally invasive method for diagnosing vaginal infections in woman by means of determining the differential or altered expression of specific proteins. The specific proteins described are also useful to predict and evaluate the efficacy of the treatment of BV using antibiotics and to identify efficacious antibiotic dosage for the cure and the remission of BV.

By the evaluation of the presence of the specific proteins found according to the methods of the invention, it is possible to predict the efficacy of the antibiotic treatment in the vaginal infections also during and before the treatment. The expression levels, or altered expression levels, of specific proteins can be used to evaluate and predict vaginal infection cure, recovery, and remission after rifaximin treatment. In addition, the methods allow the identification of the most efficacious rifaximin dosages for a given patient, as well as to the identification of those patients who are not responding to a course of treatment, e.g., a rifaximin therapy. By identifying specific proteins present in the vaginal fluid, it is possible to predict with a high percentage of success the remission of, recovery from, or elimination of the infection after antibiotic treatment, in particular rifaximin treatment. The inventive methods also provide a set of a specific proteins useful for diagnosing vaginal infections, for evaluating remission or recovery from vaginal infections, for evaluating at the time of diagnosis, the probability that a patient will enter remission upon completing antibiotic treatment, and the optimal dosage for obtaining the remission, with analytical techniques such as proteomics, Mass spectrometry, Elisa, Western blotting, Nuclear Magnetic Resonance.

According to the present methods, proteomic techniques are useful for diagnosing BV by means of analysing and/or characterizing the specific proteins identified as biomarkers. Other analytical techniques available in the art are also useful to analyze and determine the amount of the proteins such as Mass Spectrometry, Elisa, Western blotting, NMR.

Also provided is a diagnostic kit for use characterizing at least one protein useful for identifying the status of a vaginal infection. The kit includes instructions for carrying out a method of diagnosing vaginal infection using mass spectrometry. The diagnostic method of the invention has been tested on vaginal fluids collected from women enrolled in a clinical study of 80 Belgian pre-menopausal, non-pregnant women, aged between 18 and 50 years were analyzed. At the screening visit (V1) diagnosis of health or BV was made using both Amsel's criteria and Gram stain Nugent scoring. Patients with Nugent score >3 and positive for at least 3 of 4 Amsel's criteria were considered positive for BV. According to this clinical evaluation, women were divided into 2 groups: healthy subjects (H), (n=41) and patients diagnosed for BV (n=39). Patients who were diagnosed for BV at the study visit V1 were included in a multicenter, double-blind, randomised, placebo-controlled study reported in U.S. Ser. No. 13/559,013 (EudraCT: 2009-011826-32), that was performed to compare the efficacy of different doses of rifaximin vaginal tablets versus placebo for the treatment of BV. These patients underwent a randomisation visit and were distributed into four treatment groups:
group A received 100 mg rifaximin vaginal tablet once daily for 5 days (n=10),
group B received 25 mg rifaximin vaginal tablet once daily for 5 days (n=10),
group C received 100 mg rifaximin vaginal tablet once daily for the first 2 days and placebo vaginal tablet for the remaining 3 days (n=9), and
group D received placebo vaginal tablet once daily for 5 days (n=10).

After 7 to 10 days from the end of the therapy a follow-up visit (V3) was performed. Remission was evaluated at V3 according to Amsel's criteria (<3) and Gram stain Nugent score (3).

Standardized vaginal rinsings (i.e., vaginal fluid or VF) with 2 mL of saline were collected for analysis at V1 and V3, from which DNA and proteins were isolated from vaginal fluid (i.e., vaginal isolates) for further analysis.

The qPCR real-time quantitative PCR was used for the identification and quantification of bacterial groups involved in the imbalance that effects the vaginal microbiota, in particular to determine the concentrations of the principal bacterial groups which are known to be affected in the presence of BV such as Lactobacillus, Atopobium, Gardnerella vaginalis, Prevotella, Veillonella, Mobiluncus and Mycoplasma hominis.

qPCR was performed on DNA samples extracted from CVF of healthy women (H), women affected by BV at V1 (BV), women who were in remission after rifaximin treatment at V3 (R), and women who were not in remission after antibiotic or placebo treatment at V3 (N).

The molecular analysis of vaginal microbiota composition is illustrated in Table 5, wherein it is reported the percentage of women belonging of the analyzed bacterial groups in relation to the clinical status of the subject, healthy (H) or BV-affected (BV), and to the response to antibiotic treatment, remission (R) or not remission (N). Quantification of Lactobacillus, Atopobium, G. vaginalis, Prevotella, Veillonella, Mobiluncus and M. hominis are represented in Table 6. The data are expressed as ng of DNA of the target genus or species per μg of total bacterial DNA extracted from the vaginal sample in qPCR analysis.

The data confirm that Lactobacillus genus are significantly lower in BV group than in H group. After antibiotic treatment, the median value of Lactobacilli in R group is about 10 times higher than in BV group. On the contrary, the lactobacilli measured for N group was very similar to that of BV group, and significantly lower compared to both H and R groups. Atopobium concentration in R group was significantly lower than that detected in BV group even if still higher compared to H group. N group hosted significantly higher amounts of Atopobium in comparison to both H and R groups. Similarly to Atopobium, G. vaginalis and Prevotella were present in very low concentrations in H and R groups, and significantly higher concentrations were found in BV and N groups. Veillonella and Mobiluncus were not quantified in any of the women belonging to H and R groups, while few women belonging to BV and N groups hosted these bacterial groups. A significant reduction of M. hominis was found in R group compared to BV group.

Standardized vaginal fluid collected from women enrolled in the clinical study were analyzed by qualitative and quantitative proteomic techniques, e.g., qPCR, the proteins present in the vaginal fluid characterized. For example, proteins were isolated from the vaginal fluid and these vaginal isolates were optionally fractionated (e.g., chromatography) and then mass spectrometry techniques (MS/MS) were employed to detect the changes in the amount of specific proteins from one sample or sample pool compared to another. The proteomic profiles were compared to identify differences in the proteomes, for example, between healthy and diseased patients, or patients at different stages of treatment and remission.

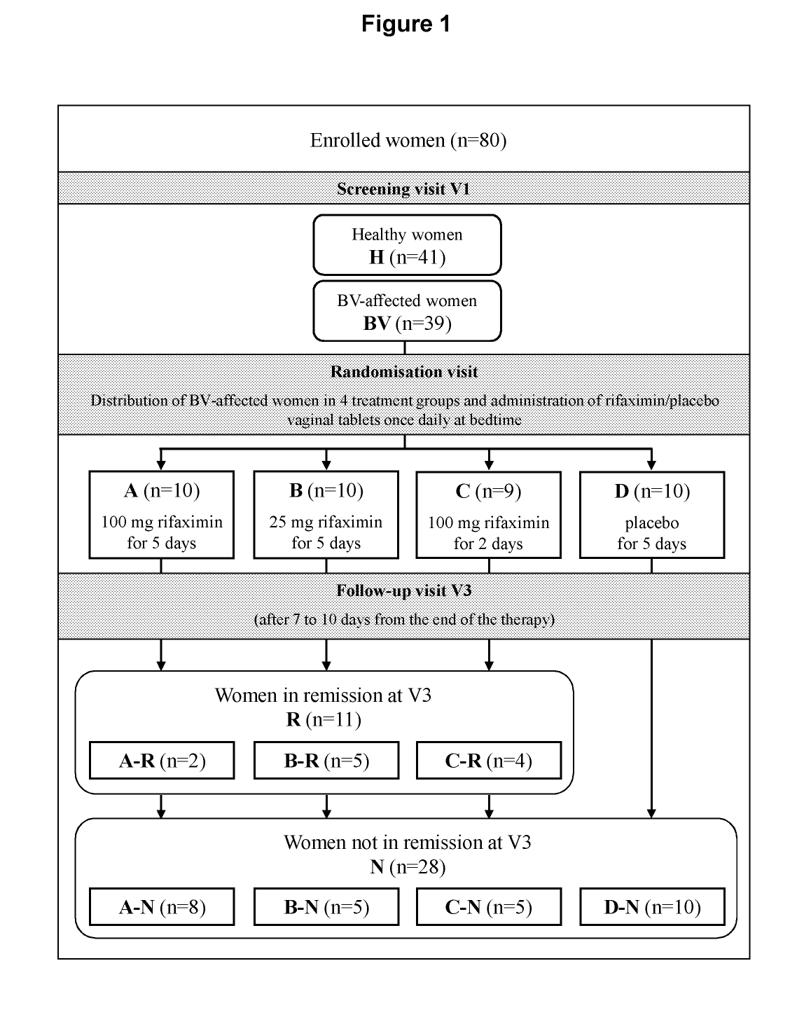

Proteomic analysis was performed on 9 pools of vaginal isolates, grouped according to the status of the BV infection:
H pool (from 41 healthy women); BV pool (from 39 BV-affected women);
A-R pool (from 2 women who were in remission after rifaximin treatment-group A);
A-N pool (from 8 women who were not in remission after rifaximin treatment-group A);
B-R pool (from 5 women who were in remission after rifaximin treatment-group B);
B-N pool (from 5 women who were not in remission after rifaximin treatment-group B);
C-R pool (from 4 women who were in remission after rifaximin treatment-group C);
C-N (from 5 women who were not in remission after rifaximin treatment-group C);
D-N (from 10 women who were not in remission after placebo treatment-group D). Scheme of the vaginal fluids collected for testing the diagnostic method is represented in FIG. 1.

A database search was conducted (Mascot search engine, database provided by Matrix Science) using the acquired mass spectrometry data, which identified a total of 131 human and microbial proteins in the fractionated pools obtained from healthy women (H) and BV affected women (BV). Interestingly, the expression change in the vast majority, i.e., about 70%, of the human proteins corresponding to the BV pool were up regulated with a median 5.5-ratio (range 1.5- to 521.1-fold). Expression changes greater than 50-fold were found for NSFL1 cofactor p47 (521.1-fold), ERO1-like protein alpha (90.2-fold), Desmoglein-3 (59.5-fold) and Glycine cleavage system H protein (53.5-fold). Of note, according to HPA, all of these proteins are moderately to strongly expressed in normal female tissues. A significant down-regulation, ranging from −1.5- to −5645.4-fold (median, −7.0), occurred for about 25% human proteins. The highest expression change was observed for calcium-activated chloride channel regulator 4 that, according to HPA, is primarily expressed in the digestive tract and present only in small amounts in urogenital organs.

Superoxide dismutase (−49.2-fold) and Serpin B4 (−43.5-fold) were also found to be significantly under-expressed.

With the data of the protein differently expressed identified by MS/MS a multivariance analysis was executed.

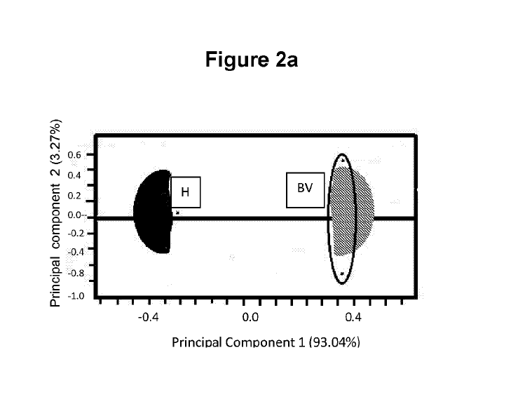

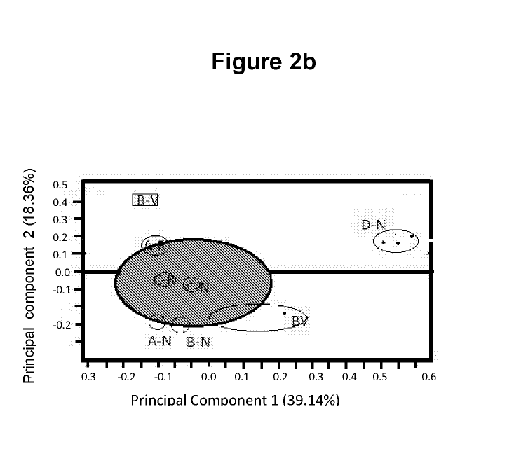

The multivariance analysis is known as Principal Component Analysis (PCA) and the result of PCA are shown in FIG. 2a and FIG. 2b. In these figures are reported the peptides in the fractionated pools of healthy women, women affected by BV and peptides of women treated with rifaximin. The treatment with rifaximin was at dosage of 100 mg/day for 5 days; (remission, A-R; no remission, A-N), 25 mg/5 days (remission, B-R; no remission, B-N), 100 mg/2 days (remission, C-R; no remission, C-N), and placebo for 5 days (D-N). Two types of comparisons were carried out: (i) between fractionated pools of proteins from vaginal fluid of healthy women (HF) versus fractionated pool of proteins from vaginal fluid of women affected by bacterial vaginosis (BVF); (ii) between whole pools of proteins from vaginal fluid of women affected by BV treated by administering different doses of rifaximin or placebo before (BV) and after (A-R, A-N, B-R, B-N, C-R, C-N, D-N) treatment. Fractioned pools of proteins are obtained, e.g., by separating the proteins by separation on membrane filtration with different membrane porosity, prior to MS/MS analysis.

Proteins identified by proteomic techniques were submitted for Gene Ontology (GO) analysis (AmiGO version 1.8, database release Mar. 11, 2012) to identify biological processes, molecular functions and subcellular localizations associated with the identified proteins. MS/MS data were further evaluated for tissue expression patterns using the publicly available Human Protein Atlas database (HPA).

Table 9, 10 and 11 report the percentage related to the Gene Ontology (GO) categorization of the MS/MS-identified proteins differentially expressed between healthy and BV-affected women. The classification was performed according to keyword categories as biological process, cellular component, molecular function. When proteins were associated with more than one functional category, one GO term was chosen arbitrarily.

Each human protein was assigned to a biological process, a cellular localization and a molecular function based on information from the GO database. The largest group of differentially expressed proteins, about 23%, were involved in the innate immune response and complement activation. Interestingly, this GO category grouped 14 immunoglobulin chain regions that, with the sole exception of Ig mu chain C region (−6.1-fold expression change), were all over-expressed in BV with a median 7.1-ratio. A marked up-regulation in BV was also observed for Complement C3 (34.5-ratio), Inter-alpha-trypsin inhibitor heavy chain H1 (36.9-ratio) and Lymphocyte-specific protein 1 (21.4-ratio), which fell into the same biological process category.

Epidermis development and keratinization accounted for 15% of the identified proteins whereas 14% were classified as involved in small molecule metabolic process. Only 5% of proteins were involved in the inflammatory response.

More than half of the dysregulated proteins were localized in the extracellular space (37%) or associated to plasma membrane (16%).

Nearly a quarter of identified proteins were cytoplasmic (23%). According to molecular function as much as 53% of the differentially expressed proteins were classified as having binding activity. Among these, protein binding (20%) was the most represented GO category, followed by calcium ion (14%) and antigen binding (13%). Twenty and 17% of identified proteins were related to enzymatic and structural molecule activity, respectively.

Pathways and networks involving differentially expressed human proteins were analyzed using MetaCore®, Thomson Reuter, an integrated software for functional analysis. Enrichment analysis revealed that the majority of enriched pathways were related to cytoskeleton remodelling, complement activation (classical, alternative and lectin-induced pathways) and blood coagulation (data not shown). To map interaction among proteins, the shortest paths were analyzed using the “analyze network” algorithm. Based on the functional sub-networks built, the proteins differentially expressed in HF and BVF pools were primarily involved in developmental process (P=1.22×10−31), immune system process (P=3.93×10−22) and response to chemical stimulus (P=1.71×10−20).

Among the 13 microbial proteins that were differentially expressed between HF and BVF pools, 9 (about 69%) were derived from Lactobacillus strains, belonging to L. acidophilus, L. casei, L. gasseri and L. helveticus species, and were mainly involved in glucose metabolism and protein synthesis. Out of these 9, 5 were down-regulated in BVF pool with a median −7.7-ratio, including 4 proteins from L. acidophilus, which is one of the main H₂O₂-producing Lactobacillus species and supports that Lactobacillus is involved in the protection of a healthy vaginal microbiota.

Among the 4 proteins over-expressed in BV, 2 were from L. gasseri, one of the most frequently occurring Lactobacillus species in vagina (median 3.2-ratio). The remaining proteins were from L. casei (n=1) and L. helveticus (n=2), species that can be found in the vaginal ecosystem as a consequence of rectovaginal cross-contamination. Interestingly, 3 enolases and 2 triosephosphate isomerases from different Lactobacillus species with contrasting expression patterns were identified, suggesting a lack of correlation between these proteins and BV condition. Three proteins from Staphylococcus aureus (Cold shock protein cspA), S. epidermidis (L-lactate dehydrogenase) and Candida glabrata (Cytoplasmic tRNA 2-thiolation protein 2) were significantly increased in BVF pool (48.0-, 3.6- and 2.7-ratio, respectively), even though none of these bacteria are known to be associated with BV. One protein from Saccharomyces cerevisiae (Transcription factor PDR8) was down-regulated in BVF pool (−36.2-ratio).

According to mass spectral analysis (MS/MS), most of the 284 human proteins identified as being differentially expressed in the vaginal fluid of women BV affected were down-regulated in patients treated with rifaximin, thus indicating the impact of rifaximin in counteracting protein profile alterations observed in BV affected women and restoring a healthy condition to the vaginal ecosystem.

Table 10 reports the percentage of the Gene Ontology (GO) categorization of the MS/MS-identified proteins differentially expressed between BV-affected women before and after rifaximin/placebo treatment. Classification was performed according to keyword categories such as (a) biological process, (b) cellular component, (c) molecular function and when proteins were associated with more than one functional category, one GO term was chosen arbitrarily.

Similar to the comparison BV versus healthy women (H), the main categories identified by GO classification are associated with the innate immune response, complement activation and small molecule metabolic process, whereas only a small percentage, less than 3%, are involved in the inflammatory response. However, immunoglobulin and other immune molecules exhibited a trend toward under-expression. This observation is contrary to what is found in the comparison of the results for the BV versus H dataset, indicating a general shutdown of immune response after antibiotic treatment.

This proteomic study also highlights the importance of the antibiotic dosage in modulating the vaginal proteome.

In the protein analysis, placebo administration is associated with the lowest number of differential proteins and the expression variation is in the opposite direction with respect to the trend observed in the rifaximin treated women.

Interestingly, pools A-N and B-N were in line with BV pool and to C-R and C-N pools, suggesting a similarity among the proteomic profiles of BV-affected women, women who were not in remission after rifaximin treatment and women who received the antibiotic only for two days.

The largest number of differentially expressed proteins was identified following administration with 25 mg of rifaximin once daily for 5 days, dosage that induced also the highest fold changes in protein expression, thus further confirming the major impact of this treatment regimen onto BV-related proteome.

Some specific proteins are meaningful in order to assess bacterial infections. Table 1 and Table 2 present a set of significant proteins obtained from Table 7 and Table 8 selecting the most significant proteins present in the vaginal fluid sampled from women affected by a vaginal infection versus a reference sample representing vaginal fluid sampled from healthy women, which are meaningful in order to diagnose and evaluate the status of infections, e.g., bacterial vaginosis.

These specific proteins are influenced by bacterial vaginosis (BV) and are useful in the diagnosis of BV are thus referred to as “specific biomarkers for BV”. Table 1 presents a set of significant proteins that increase in BV affected women versus a reference sample representing vaginal fluid sampled from healthy women, e.g., non infected women, while Table 2 presents a set of significant proteins which decrease in the BV affected women versus a reference sample representing vaginal fluid sampled from healthy women. Examples of specific proteins that decrease or increase with respect to the health condition or state of an infection (e.g., BV) are: Vitamin D binding protein, Desmocollin-2, Calcium-activated chloride channel regulator 4, Catalase, Small proline-rich protein 3, Galectin-3-binding protein, Hemopexin, Immunoglobulin family, Intermediate filament family, Lipocalin family, Alpha 1-acid glycoprotein 1, Alpha-1-acid glycoprotein 2, Neutrophil gelatinase-associated lipocalin, Limphocyte-specific protein 1, Myeloblastin, Perilipin-3, Perilplakin, Protein S100-A9, Protein S100-A7, Superoxide dismutase [Cu—Zn].

Changes in the amounts of the specific proteins present in the vaginal fluid sampled from a patient affected by a vaginal infection versus a reference sample representing vaginal fluid sampled from healthy women, are diagnostic for determining the presence of an infection, e.g., bacterial vaginosis.

For example, when a differentially expressed protein is at least one of the specific proteins identified in Table 1 and has a ratio greater than 1.5, a bacterial infection is positively diagnosed. Preferably, at least two specific proteins identified in Table 1 have a ratio greater than 1.5. More preferably, three or more of the specific proteins identified in Table 1 have a ratio greater than 1.5. In some embodiments of the inventive methods, the ratio is greater than 3, preferably greater than 5, 10 or 20.

In another example, when a differentially expressed protein is at least one of the specific proteins identified in Table 2 and has a ratio less than −1.5, a bacterial infection is positively diagnosed. Preferably, at least two specific proteins identified in Table 2 have a ratio less than −1.5. More preferably, three or more of the specific proteins identified in Table 2 have a ratio less than −1.5. In some embodiments of the above methods, the ratio less than −3, preferably, greater than −5, −10 or −20. In a particular example, the a bacterial infection is positively diagnosed by a reduction in Calcium-activated chloride channel regulator 4 with a ratio of less than −5000, preferably, less than −5500.

TABLE 1

| Protein name | Ratio: BV/H |
|---|---|
| Desmocollin-2 | 8.9 |
| Small proline-rich protein 3 | 2.0 |
| Immunoglobulin J chain | 3.7 |
| Intermediate filament family | |
| Keratin, type I cytoskeletal 10 | 30.3 |
| Keratin, type II cytoskeletal 1 | 4.9 |
| Keratin, type II cytoskeletal 2 epidermal | 39.8 |
| Keratin, type II cytoskeletal 5 | 2.0 |
| Neutrophil gelatinase-associated lipocalin | 19.7 |
| Lymphocyte-specific protein 1 | 21.4 |
| Perilipin-3 | 1.9 |
| Periplakin | 3.5 |

TABLE 2

| Protein name | Ratio: BV/H |
|---|---|
| Vitamin D binding protein | −2.8 |
| Calcium-activated chloride channel regulator 4 | −5645.4 |
| Catalase | −16.2 |
| Galectin-3-binding protein | −8.8 |
| Hemopexin | −1.7 |
| Immunoglobulin family | |
| Ig mu chain C region | −6.1 |
| Lipocalin family | |
| Alpha-1-acid glycoprotein 1 | −7.9 |
| Alpha-1-acid glycoprotein 2 | −28.6 |
| Myeloblastin | −5.1 |
| Protein S100-A9 | −4.0 |
| Protein S100-A7 | −4.3 |
| Superoxide dismutase [Cu—Zn] | −49.2 |

Table 3 and 4 show the protein ratio in the vaginal fluid of the BV affected women (BV) versus the women in remission (R) after treatment with rifaximin at different dosages and different times of treatment.

Table 3 shows the decreasing of the proteins after treatment with different rifaximin dosage in the following comparison: BV-affected woman (BV) versus (R) induced by different dosages of rifaximin (A-R, B-R, C-R).

Changes in the amounts of the specific proteins present in the vaginal fluid samples from a patient affected by a vaginal infection, e.g., bacterial vaginosis, versus vaginal fluid sampled from a patient after treatment, are diagnostic for evaluating the status of infections, for example if the infection is persisting and the patient is a non responder to the treatment, if the infection is in remission or if the infection is cured.

For example, when a differentially expressed protein is at least one of the specific proteins identified in Table 3 and has a ratio greater than 1.5, remission of a bacterial infection is positively determined. Preferably, at least two specific proteins identified in Table 1 have a ratio greater than 1.5. More preferably, three or more of the specific proteins identified in Table 1 have a ratio greater than 1.5. In some embodiments of the inventive methods, the ratio is greater than 2, preferably, greater than 3, 5 or 10. In a particular embodiment, remission is determined by the increase in Hemopexin or Protein S100-A7 by a ratio greater than 1.5.

In another example, when a differentially expressed protein is at least one of the specific proteins identified in Table 4 and has a ratio less than −1.5, remission of a bacterial infection is positively determined. Preferably, at least two specific proteins identified in Table 2 have a ratio less than −1.5. More preferably, three or more of the specific proteins identified in Table 2 have a ratio less than −1.5. In some embodiments of the above methods, the ratio is less than −3, preferably, greater than −5, −10 or −20. In a particular example, remission of a bacterial infection is positively determined by a reduction in Small proline-rich protein 3, Perilipin-3, Periplakin and/or Immunoglobulin J chain in an ratio of less than −1.5, preferably, less than −2, more preferably, less than −3.

Differences in the amounts of the specific proteins present in the vaginal fluid sampled from a pool of patients affected by a vaginal infection after various treatments, are useful for identifying the most efficacious treatment for evaluating the status of infections. In general, the efficacy of a treatment can be evaluated by the total number of differentially expressed proteins (determined by comparison of the proteome profiles before and after treatment) for a specific treatment. The greater the number of differentially expressed proteins, in particular those identified in Tables 3 and 4, the greater the efficacy of the treatment.

TABLE 3

| | Ratio: BV/R | | |
|---|---|---|---|
| | A-R | | C-R |
| | 100 mg × | B-R | 100 mg × |
| Protein name | 5 days | 25 mg × 5 days | 2 days |
| Vitamin D-binding protein | 1.7 | 17.8 | 0.0 |
| Calcium-activated chloride | 2.9 | 0.0 | 1.8 |
| channel regulator 4 | | | |
| Catalase | 0.0 | 4.0 | 2.3 |
| Galectin-3-binding protein | 3.6 | 2.7 | 0.0 |
| Hemopexin | 1.9 | 9.0 | 1.7 |
| Immunoglobulin family | | | |
| Ig mu chain C region | 1.8 | 8.1 | 0.0 |
| Lipocalin family | | | |
| Alpha-1-acid glycoprotein 1 | 2.3 | 7.8 | 0.0 |
| Alpha-1-acid glycoprotein 2 | 1.5 | 19.5 | −1.9 |
| Myeloblastin | 0.0 | 3.1 | 4.0 |
| Protein S100-A9 | 4.2 | 2.6 | 0.0 |
| Protein S100-A7 | 19.2 | 5.7 | 2.5 |
| Superoxide dismutase [Cu—Zn] | 1.8 | 1.5 | 0.0 |

Table 4 presents the proteins that increase after treatment with rifaximin in the following comparison: BV versus (R) induced by different dosages of rifaximin (A-R, B-R, C-R).

TABLE 4

| | Ratio: BV/R | | |
|---|---|---|---|
| | A-R | | C-R |
| | 100 mg × | B-R | 100 mg × |
| Protein name | 5 days | 25 mg × 5 days | 2 days |
| Desmocollin-2 | −1.5 | −2.0 | 0.0 |
| Small proline-rich protein 3 | −6.6 | −6.6 | −4.4 |
| Immunoglobulin family | | | |
| Immunoglobulin J chain | −1.5 | −4.1 | −2.2 |
| Intermediate filament family | | | |
| Keratin, type I cytoskeletal 10 | −2.8 | −3.5 | 0.0 |
| Keratin, type II cytoskeletal 1 | −3.9 | −6.2 | −2.3 |
| Keratin, type II cytoskeletal 2 | −4.3 | −7.1 | 0.0 |
| epidermal | | | |
| Keratin, type II cytoskeletal 5 | −16.3 | −12.7 | −2.5 |
| Lipocalin family | | | |
| Neutrophil gelatinase- | 0.0 | −1.5 | 0.0 |
| associated lipocalin | | | |
| Lymphocyte-specific protein 1 | −2.8 | −3.2 | 0.0 |
| Perilipin-3 | −1.7 | −2.9 | −2.3 |
| Periplakin | −3.6 | −3.4 | −2.0 |

The clinical study described in U.S. Ser. No. 13/559,613, incorporated by reference herein in its entirety, reports that group B obtained remission from the infection after treatment with 25 mg rifaximin for 5 days. One aspect of the presently described methods is to evaluate the efficacy of treatment of BV, for example, identifying remission of BV is indicated by the change in the expression of specific proteins as described herein. Another aspect is to evaluate the patient's response to antibiotic treatment, in particular, to rifaximin therapy. The identification of non-responder patients is particularly important so that the treatment can be modified, either by changing the dosage or by changing the antibiotic therapy to produce a positive clinical response in which the BV is in remission.

Tables 3 and 4 show that Vitamin D-binding protein, Immunoglobulin family, Lipocalin family, Myeloblastin family, are preferred specific biomarkers for the evaluation of the remission of BV after rifaximin treatment, also preferred are the specific proteins in the Immunoglobulin family, Ig mu chain C region and Immunoglobulin J chain; in the Lipoclin family, Alpha-1-acid glycoprotein 1, Alpha-1-acid glycoprotein 2; in the Intermediate filament family, Keratine tipe II cytoskeletal 1, the Keratine tipe II cytoskeletal 2 epidermal and Keratine tipe II cytoskeletal 5.

According to the disclosure of the present methods, proteomic techniques are useful for diagnosing BV by means of analysing and/or characterizing the specific proteins identified as biomarkers. Other analytical techniques available in the art are also useful to analyze and determine the amount of the proteins such as Elisa, Western blotting, NMR.

Also provided is a diagnostic kit for use characterizing at least one protein useful for identifying a vaginal infection. The kit includes instructions for carrying out a method of diagnosing vaginal infection using mass spectrometry.

The Example 1 describes the real time PCR based upon the sequence analysis of DNA and showing the microbial composition of the vaginal ecosystem of samples collected in healthy and BV affected women.

The Example 2 describes the determination of the proteins, (proteomic profile) present in the vaginal fluid using mass spectrometry and Table 7 reports proteins which are differentially expressed between healthy women (HF) and BV-affected women (BVF) as identified by mass spectrometry analysis.

Table 8 reports proteins which are differentially expressed between BV affected women before (BV) and after (A-R, A-N, B-R, B-N, C-R, C-N, D-N) treatment as identified by mass spectrometry analysis.

Following rifaximin and placebo treatment 284 human proteins were identified as present in vaginal fluid from BV affected women, 48 (about 17%) were present in all pools from rifaximin-treated women compared to BV pool, regardless of both antibiotic dosage and clinical outcome. In particular, 23 proteins increased and 17 decreased after treatment, whereas contrasting variations in protein abundance were observed for the remaining 8 proteins. Notably, increases of several hundred-up to over a thousand-fold were found for Keratin type II cytoskeletal 74 (range 789.6- to 13424.4-fold), protein FAM25 (range 437.6- to 8944.5-fold) and Werner syndrome ATP-dependent helicase (range 12.4- to 750.5-fold) in rifaximin treatment groups. Interestingly, the highest variations for these proteins occurred in B-R, followed by A-R pool, while little or no changes were observed after placebo administration. According to HPA, all three proteins are moderately to strongly expressed in both female tissues and digestive tract. Noteworthy, protein FAM25 had been previously identified as significantly down-regulated (−11.2-ratio) in BVF respect to HF pool. Similar opposite trends were obtained for the other 45 of the 89 proteins that were differentially expressed in both proteomic comparison datasets. In particular, 25 of these proteins were up-(5) or down-regulated (20) in at least 4 of the 6 pools from rifaximin-treated women, contrary to what was found in BV versus H comparison. For 17 out of 25 (68%) proteins, the highest ratios were associated with B-R pool. Interestingly, group B showed the largest total number of differentially expressed human proteins with 214 and 155 differentially expressed proteins in B-R and B-N pools, respectively. Moreover, the fold changes of 83 proteins in B-R pool were the highest among pools, suggesting a major impact of this treatment regimen onto BV-related proteome. In particular, in addition to Keratin type II cytoskeletal 74, protein FAM25 and Werner syndrome ATP-dependent helicase, expression changes greater than 50-fold were found for Stanniocalcin-1 (113.1-ratio), Kininogen-1 (−88.6-ratio) and Prostate stem cell antigen (63.8-ratio). Zinc-alpha-2-glycoprotein (−9.4-ratio), Ig heavy chain V-III region BUT (−1.6-ratio) and VH26 (−1.5-ratio), Kallikrein-13 (1.5-ratio) and Neutrophil gelatinase-associated lipocalin (1.5-ratio) were identified as differentially expressed only in B-R pool. Of note, 174 proteins were shared between A-R and B-R pools, and 168 (97%) exhibited the same trend of expression. Conversely, only 138 proteins were common to B-R and C-R pools and 24 (17%) had opposite fold changes.

Placebo administration was associated with the lowest number of differential proteins (207). Expression changes over 50-fold in D-N pool were found for protein NDRG1 (−1317.2-ratio), Ig lambda-7 chain C region (−957.1-ratio), protein S100-P (−443.4-ratio), Leucine-rich repeat-containing protein 8E (−205.8-ratio), Ig kappa chain V-III region POM (−83.6-ratio) and Immunoglobulin J chain (−50.8-ratio). Notably, except for Ig kappa chain V-III region POM, the protein expression variation was in the opposite direction with respect to the trend observed in the other pools.

Each of the differentially expressed human proteins identified were assigned to a biological process, a cellular localization and a molecular function based on information from the GO database. Similarly to the comparison BV versus H, most proteins were involved in the innate immune response and complement activation (22%) and small molecule metabolic process (16%), whereas only 3% were involved in the inflammatory response. Interestingly, in the most represented GO category, only about 14% proteins increased after rifaximin treatment, 32 (54%) decreased whilst contrasting variations were found for 19 (32%) proteins.

Of note, this category grouped 17 proteins that were identified as differentially expressed also in BV respect to H pool. Ten of these proteins, namely, Annexin A3, Complement C3, Ig gamma-2 chain C region, Ig heavy chain V-III region VH26, Ig kappa chain C region, Ig kappa chain V-IV region (Fragment), Ig lambda chain V-III region LOI, Ig lambda chain V-IV region Hil, Ig lambda-1 chain C regions, and Lactotransferrin, exhibited a trend toward under-expression, contrary to what was found in BV versus H dataset. A large amount of proteins were localized in with the extracellular space (39%) and plasmatic membrane (12%). As much as 20% of the differentially expressed proteins were cytoplasmic. The main represented molecular functions were structural molecule activity (19%), antigen binding (15%) and protein binding (14%).

Pathways and networks involving the differentially expressed human proteins were analyzed using MetaCore™ database search, Thompson Reuters. According to the enrichment analysis, the most enriched pathways were associated with cytoskeleton remodelling, blood coagulation and complement activation, similarly to the previous analysis of HF and BVF pools.

More than half, i.e., about 53%, of the 30 microbial proteins that were differentially expressed in BV, A-R, A-N, B-R, B-N, C-R, C-N and D-N pools were from Lactobacillus species (L. acidophilus, L. brevis, L. casei, L. delbrueckii subsp. bulgaricus, L. gasseri, L. helveticus, L. johnsonii), and were mainly involved in glucose metabolism, replication and protein synthesis. Interestingly, only trigger factor from L. brevis was found to be down-regulated in all pools after rifaximin treatment with a median −2.5-ratio. Six Lactobacillus proteins were over-(2) or under-expressed (4) in at least 2 of the 6 pools from antibiotic-treated women, whereas Pyruvate kinase (1.5-ratio) and Triosephosphate isomerase (2.4-ratio) from L. delbrueckii subsp. bulgaricus were affected only in A-R and B-N pool, respectively. Contrasting expression patterns among pools were observed for the remaining 7 proteins from Lactobacilli. Notably, 4 enolases from L. acidophilus, L. delbrueckii subsp. bulgaricus, L. gasseri and L. helveticus were identified, but only the first 3 exhibited a trend of down-regulation in response to antibiotic administration, with a median −2.7-ratio. The maximum fold change was observed in A-R pool for Phosphoglycerate kinase from L. gasseri (−22.1-fold), but the protein was found to be over-expressed in A-N, B-R, C-R and D-N pools, suggesting a lack of correlation with the antibiotic treatment.

Fourteen (47%) differentially expressed microbial proteins were from other microorganisms that can be associated with the vaginal environment, namely: Oenococcus oeni, Pichia guilliermondii, Bifidobacterium longum subsp. infantis, S. cerevisiae, S. epidermidis, Ureaplasma parvum, Mycoplasma genitalium, Escherichia coli and S. aureus. In particular, Phosphoglycerate kinase and probable DNA helicase II homolog were from U. parvum and M. genitalium, respectively, which are known to be associated with BV. Three proteins were also identified in one or more Lactobacillus species, but with contrasting fold changes: 60 kDa chaperonin (L. gasseri and O. oeni), Phosphoglycerate kinase (L. gasseri, L. helveticus and U. parvum) and Pyruvate kinase (L. delbrueckii subsp. bulgaricus and S. aureus). Phosphoglycerate kinase from U. parvum (median 5.1-ratio) and UPF0082 protein SAB0618 from S. aureus (median 8.9-ratio) were up-regulated in all pools, while a median −2.3-fold down-regulation was observed for Malate dehydrogenase from S. cerevisiae.

Table 8 presents the proteins which are differentially expressed between BV affected women before (BV) and after (A-R, A-N, B-R, B-N, C-R, C-N, D-N) treatment as identified by mass spectrometry analysis.

Following rifaximin and placebo treatment 284 human proteins were identified as differentially expressed in CV from BV affected women, 48 (about 17%) were differentially expressed in all pools from rifaximin-treated women compared to BV pool, regardless of both antibiotic dosage and clinical outcome. In particular, 23 proteins increased and 17 decreased after treatment, whereas contrasting variations in protein abundance were observed for the remaining 8 proteins. Notably, increases of several hundred-up to over a thousand-fold were found for Keratin type II cytoskeletal 74 (range 789.6- to 13424.4-fold), protein FAM25 (range 437.6- to 8944.5-fold) and Werner syndrome ATP-dependent helicase (range 12.4- to 750.5-fold) in rifaximin treatment groups. Interestingly, the highest variations for these proteins occurred in B-R, followed by A-R pool, whilst little or no changes were observed after placebo administration.

According to HPA, all three proteins are moderately to strongly expressed in both female tissues and digestive tract. Noteworthy, protein FAM25 had been previously identified as significantly down-regulated (−11.2-ratio) in BV respect to H pool. Similar opposite trends were obtained for the other 45 of the 89 proteins that were differentially expressed in both proteomic comparison datasets. In particular, 25 of these proteins were up-(5) or down-regulated (20) in at least 4 of the 6 pools from rifaximin-treated women, contrary to what was found in BV versus H comparison. For 17 out of 25 (68%) proteins, the highest ratios were associated with B-R pool. Interestingly, group B showed the largest total number of differentially expressed human proteins with 214 and 155 differentially expressed proteins in B-R and B-N pools, respectively. Moreover, the fold changes of 83 proteins in B-R pool were the highest among pools, suggesting a major impact of this treatment regimen onto BV-related proteome. In particular, in addition to Keratin type II cytoskeletal 74, protein FAM25 and Werner syndrome ATP-dependent helicase, expression changes greater than 50-fold were found for Stanniocalcin-1 (113.1-ratio), Kininogen-1 (−88.6-ratio) and Prostate stem cell antigen (63.8-ratio). Zinc-alpha-2-glycoprotein (−9.4-ratio), Ig heavy chain V-III region BUT (−1.6-ratio) and VH26 (−1.5-ratio), Kallikrein-13 (1.5-ratio) and Neutrophil gelatinase-associated lipocalin (1.5-ratio) were identified as differentially expressed only in B-R pool. Of note, 174 proteins were shared between A-R and B-R pools, and 168 (97%) exhibited the same trend of expression. Conversely, only 138 proteins were common to B-R and C-R pools and 24 (17%) had opposite fold changes.

Placebo administration was associated with the lowest number of differential proteins (207). Expression changes over 50-fold in D-N pool were found for protein NDRG1 (−1317.2-ratio), Ig lambda-7 chain C region (−957.1-ratio), protein S100-P (−443.4-ratio), Leucine-rich repeat-containing protein 8E (−205.8-ratio), Ig kappa chain V-III region POM (−83.6-ratio) and Immunoglobulin J chain (−50.8-ratio). Notably, except for Ig kappa chain V-III region POM, the protein expression variation was in the opposite direction with respect to the trend observed in the other pools.

Each of the differentially expressed human proteins identified were assigned to a biological process, a cellular localization and a molecular function based on information from the GO database. Similarly to the comparison BV versus H, most proteins were involved in the innate immune response and complement activation (22%) and small molecule metabolic process (16%), whereas only 3% were involved in the inflammatory response. Interestingly, in the most represented GO category, only about 14% proteins increased after rifaximin treatment, 32 (54%) decreased whilst contrasting variations were found for 19 (32%) proteins.

Of note, this category grouped 17 proteins that were identified as differentially expressed also in BV respect to HF pool. Ten of these proteins, namely, Annexin A3, Complement C3, Ig gamma-2 chain C region, Ig heavy chain V-III region VH26, Ig kappa chain C region, Ig kappa chain V-IV region (Fragment), Ig lambda chain V-III region LOI, Ig lambda chain V-IV region Hil, Ig lambda-1 chain C regions, and Lactotransferrin, exhibited a trend toward under-expression, contrary to what was found in BV versus H dataset. A large amount of proteins were localized in the extracellular space (39%) and plasmatic membrane (12%). As much as 20% of the differentially expressed proteins were cytoplasmic. The main represented molecular functions were structural molecule activity (19%), antigen binding (15%) and protein binding (14%).

Pathways and networks involving the differentially expressed human proteins were analyzed using MetaCore™ database search, Thompson Reuters. According to the enrichment analysis, the most enriched pathways were associated with cytoskeleton remodelling, blood coagulation and complement activation, similarly to the previous analysis of H and in BV, A-R, A-N, B-R, B-N, C-R, C-N and D-N pools were from Lactobacillus species (L. acidophilus, L. brevis, L. casei, L. delbrueckii subsp. bulgaricus, L. gasseri, L. helveticus, L. johnsonii), and were mainly involved in glucose metabolism, replication and protein synthesis. Interestingly, only trigger factor from L. brevis was found to be down-regulated in all pools after rifaximin treatment with a median −2.5-ratio. Six Lactobacillus proteins were over-(2) or under-expressed (4) in at least 2 of the 6 pools from antibiotic-treated women, whereas Pyruvate kinase (1.5-ratio) and Triosephosphate isomerase (2.4-ratio) from L. delbrueckii subsp. bulgaricus were affected only in A-R and B-N pool, respectively. Contrasting expression patterns among pools were observed for the remaining 7 proteins from Lactobacilli. Notably, 4 enolases from L. acidophilus, L. delbrueckii subsp. bulgaricus, L. gasseri and L. helveticus were identified, but only the first 3 exhibited a trend of down-regulation in response to antibiotic administration, with a median −2.7-ratio. The maximum fold change was observed in A-R pool for Phosphoglycerate kinase from L. gasseri (−22.1-fold), but the protein was found to be over-expressed in A-N, B-R, C-R and D-N pools, suggesting a lack of correlation with the antibiotic treatment.

Fourteen (47%) differentially expressed microbial proteins were from other microorganisms that can be associated with the vaginal environment, namely: Oenococcus oeni, Pichia guilliermondii, Bifidobacterium longum subsp. infantis, Saccharomyces cerevisiae, Saccharomyces epidermidis, Ureaplasma parvum, Mycoplasma genitalium, Escherichia coli and Staphylococcus aureus. In particular, Phosphoglycerate kinase and probable DNA helicase II homolog were from Ureaplasma parvum and Mycoplasma genitalium, respectively, which are known to be associated with BV. Three proteins were also identified in one or more Lactobacillus species, but with contrasting fold changes: 60 kDa chaperonin (Lactobacillus gasseri and acidophilus), Phosphoglycerate kinase (Lactobacillus gasseri, Lactobacillus helveticus and Ureaplasma parvum) and Pyruvate kinase (Lactobacillus delbrueckii subsp. bulgaricus and Staphylococcus aureus). Phosphoglycerate kinase from Ureaplasma parvum (median 5.1-ratio) and UPF0082 protein SAB0618 from Staphylococcus aureus (median 8.9-ratio) were up-regulated in all pools, while a median −2.3-fold down-regulation was observed for Malate dehydrogenase from Saccharomyces cerevisiae.

One embodiment of the invention is a method of diagnosing a vaginal bacterial infection in an individual undergoing testing for such infection comprising subjecting a vaginal fluid sample obtained from the individual to proteomic analysis; and determining the proteins having altered levels of expression in the test fluid sample compared with the levels of expression of the proteins in a vaginal fluid sample from a healthy or uninfected individual, wherein a decrease or increase in expression levels of proteins in the test versus the healthy sample diagnose the vaginal infection. The increase or decrease of the specified protein is a ratio preferably greater than the absolute value of 1.5, 2, 3, 4, 5, 10, 15 or 20.

One embodiment is a method of diagnosing a vaginal bacterial infection wherein the proteins which decrease or increase in the test sample versus the healthy sample are selected form the group consisting of Vitamin D binding protein, Desmocollin-2, Calcium-activated chloride channel regulator 4, Catalase, Small proline-rich protein 3, Galectin-3-binding protein, Hemopexin, Immunoglobulin family, Intermediate filament family, Lipocalin family, Alpha 1-acid glycoprotein 1, Alpha-1-acid glycoprotein 2, Neutrophil gelatinase-associated lipocalin, Limphocyte-specific protein 1, Myeloblastin, Perilipin-3, Perilplakin, Protein S100-A9, Protein S100-A7, and Superoxide dismutase [Cu—Zn].

Another embodiment is a method of diagnosis, wherein the proteins which increase in the test sample fluid versus the healthy sample fluid are selected from Desmocollin-2, Small proline-rich protein 3, Immunoglobulin J chain, keratin type I cytoskeletal 10, keratin type II cytoskeletal 1, keratin type II cytoskeletal 2 epidermal, keratin type II cytoskeletal 5, Neutrophil gelatinase-associated lipocalin, Limphocyte-specific protein 1, Perilipin-3, Perilplakin, or combinations thereof.

Another embodiment is a method of diagnosis of vaginal infection, wherein the proteins which decrease in the test sample fluid versus the healthy sample fluid are selected from Vitamin D binding protein, Calcium-activated chloride channel regulator 4, Catalase, Galectin-3-binding protein, Hemopexin, IgM chain constant region, alpha-1-acid glycoprotein 1, alpha-1-acid glycoprotein 2, Myeloblastin, Protein S100-A9, Protein S100-A7, Superoxide dismutase [Cu—Zn], or combinations thereof,

In particular, in the method of diagnosis of vaginal infection, the increase in the ratio of protein expression between the test sample and reference sample is in the range from about 1.5 to about 40 or the decrease in the ratio of protein expression between the test sample and reference sample is in the range from about −1.5 to about −5650.

In one particular embodiment is a method of diagnosis of vaginal infection, wherein the proteins which decrease in the test sample fluid versus the BV infected sample fluid after antibiotic treatment are selected from Vitamin D binding protein, Calcium-activated chloride channel regulator 4, Catalase, Galectin-3-binding protein, Hemopexin, Immunoglobulin M chain C region, Alpha 1-acid glycoprotein 1, Alpha-1-acid glycoprotein 2, Protein S100-A9, Protein S100-A7, Superoxide dismutase [Cu—Zn], or combinations thereof.

In one particular embodiment is a method of diagnosis of vaginal infection, wherein the proteins which increase in the test sample fluid versus the BV infected sample fluid after antibiotic treatment are selected from Desmocollin-2, Small proline-rich protein 3, Immunoglobulin J chain, Keratin, type I cytoskeletal 10, Keratin, type II cytoskeletal 1, Keratin, type II cytoskeletal 2 epidermal, Keratin, type II cytoskeletal 5, Neutrophil gelatinase-associated lipocalin, Lymphocyte-specific protein 1, Perilipin-3, Periplakin, or combinations thereof.

In one particular embodiment is a method of diagnosis of vaginal infection, and wherein a method of treating the diagnosed infection is by administering rifaximin.

In one particular embodiment is a method of diagnosis for evaluationg the efficacy of the rifaximin treatment before the treatment.

In one particular embodiment is a method of diagnosis for evaluating if the patients affected by BV will be or will be not in remission during and before the rifaximin treatment.

### Example 1

This Example describes the real time PCR based upon the sequence analysis of DNA and shows the microbial composition of the vaginal ecosystem of samples collected in healthy and BV affected women.

Real-Time PCR Analysis of Vaginal Bacterial Communities.

a) Sample collection

A total of 80 Belgian pre-menopausal, non-pregnant women, aged between 18 and 50 years were included in the present study. At the screening visit (V1) diagnosis of health or BV was made using both Amsel's criteria and Gram stain Nugent scoring. Patients with Nugent score >3 and positive for at least 3 of 4 Amsel's criteria were considered positive for BV. According to this clinical evaluation, women were split into 2 groups: healthy subjects (H), who had no signs of vaginal tract infection (n=41) and patients affected by BV (n=39).

Patients who were diagnosed for BV at the study visit V1 were included in a multicentre, double-blind, randomised, placebo-controlled study (EudraCT: 2009-011826-32), that was performed to compare the efficacy of different doses of rifaximin vaginal tablets versus placebo for the treatment of BV. The patients underwent a randomization visit and were distributed into 4 treatment groups: group A received 100 mg rifaximin vaginal tablet once daily for 5 days (n=10), group B received 25 mg rifaximin vaginal tablet once daily for 5 days (n=10), group C received 100 mg rifaximin vaginal tablet once daily for the first 2 days and placebo vaginal tablet for the remaining 3 days (n=9), group D received placebo vaginal tablet once daily for 5 days (n=10). Study medication was administered intra-vaginally at bedtime. After 7 to 10 days from the end of the therapy a follow-up visit (V3) was performed. Remission was evaluated at V3 according to Amsel's criteria (<3) and Gram stain Nugent score (≦3) (Table 1).

Standardized vaginal rinsings with 2 mL of saline were collected for analysis at V1 and V3 by flushing and re-aspirating the fluid through a 22 Gauge needle in the left, central and right upper vaginal vaults. The vaginal rinsings were subsequently stored at −80° C. until use.

Sample collecting is also represented in FIG. 1.

b) DNA and Protein Extraction

One mL of each vaginal rinsing was centrifuged at 9500 g for 15 min to separate the pellet, which was processed for bacterial DNA isolation, from the supernatant, used for protein extraction.

DNA amount was quantified using NanoDrop ND-1000 (NanoDrop® Technologies, Wilmington, Del.).

Nine volumes of acetone: HCl (10:1) were added to the supernatant of the vaginal rinsing and proteins were precipitated by centrifuging at 12000 g for 10 min. The protein pellet was dissolved in 1 mL of 70% ethanol and the sample was spun at 12000 g for 10 min. One mL of acetone was added and the proteins were further precipitated by centrifugation at 12000 g for 5 min. After removing supernatant, pellet was dried by SpeedVac concentrator (Thermo Savant ISS110, Thermo Fisher Scientific, Waltham, Mass.) and then stored at −20° C.

Protein extract was quantified using the 2-D Quant Kit (GE Healthcare, Uppsala, Sweden) according to the manufacturer's instructions.

c) Real Time PCR

Real-time PCR was performed on DNA samples extracted from cervicovaginal fluid (CVF) collected from 41 healthy women (H) and 39 BV-affected women before (BV) and after rifaximin/placebo treatment R (11 women in remission) and N (28 women not in remission).

Specific primer sets targeted to 16S rRNA gene or 16S-23S rRNA spacer region were used to quantify the following genus or species: Lactobacillus, Gardnerella vaginalis, Atopobium, Prevotella, Veillonella, Mycoplasma hominis and Mobiluncus.

Distribution of the majority of the target genera and species was similar in R and H groups, while N group showed a very similar profile to BV group, suggesting the efficacy of rifaximin in restoring a healthy-like condition. Table 5 reports the percentage of women belonging to the study groups H, BV, R or N, hosting each of the analysed bacterial groups.

TABLE 5

% of women

| | H | BV | R | N |
|---|---|---|---|---|
| Lactobacillus | 100 | 100 | 100 | 89.3 |
| Gardnerella vaginalis | 9.8 | 97.4 | 45.5 | 85.7 |
| Atopobium | 56.1 | 100 | 100 | 100 |
| Prevotella | 68.3 | 97.4 | 81.8 | 89.3 |
| Veillonella | 0.0 | 46.2 | 0.0 | 35.7 |
| Mycoplasma hominis | 61 | 82.1 | 63.6 | 71.4 |
| Mobiluncus | 2.4 | 17.9 | 0.0 | 10.7 |

The median concentration of Lactobacillus, Atopobium, Gardnerella. vaginalis, Prevotella, Veillonella, Mobiluncus and Mobilincus hominis in women belonging to the study groups H, BV, R and N are represented in Table 4. Data were expressed as ng of DNA of the targeted genus or species per μg of total DNA extracted from the vaginal sample.

TABLE 6

| | Target DNA/genomic vaginal DNA (ng/μg) | | | | | | |
|---|---|---|---|---|---|---|---|
| | (median ± SD) | | | | | | |
| | | | G. | | | | Mobilincus |
| Group | Lactobacillus | Atopobium | vaginalis | Prevotella | Veillonella | Mobiluncus | hominis |
| H | 1.93E+01 | 5.12E−01 | 0.0 | 3.61E−02 | 0.0 | 0.0 | 1.92E−02 |
| BV | 5.20E+00 | 1.16E+02 | 5.40E+01 | 1.32E+01 | 0.0 | 0.0 | 1.03E−02 |
| R | 4.63E+01 | 4.86E+00 | 0.0 | 4.51E−02 | 0.0 | 0.0 | 1.56E−03 |
| N | 5.16E+00 | 4.18E+01 | 4.85E+01 | 1.73E+00 | 0.0 | 0.0 | 2.50E−03 |

### Example 2

### Proteomic Analysis of Vaginal Fluid

This Example describes the determination of the proteins, (proteomic profile) present in the vaginal fluid using mass spectrometry. Table 7 presents proteins which are differentially expressed between healthy women (H) and BV-affected women (BV) as identified by mass spectrometry analysis.

a) Sample Collection

A total of 80 Belgian pre-menopausal, non-pregnant women, aged between 18 and 50 years were included in the present study. At the screening visit (V1) diagnosis of health or BV was made using both Amsel's criteria and Gram stain Nugent scoring. Patients with Nugent score >3 and positive for at least 3 of 4 Amsel's criteria were considered positive for BV. According to this clinical evaluation, women were split into 2 groups: healthy subjects (H), who had no signs of vaginal tract infection (n=41) and patients affected by BV (n=39).

Patients who were diagnosed for BV at the study visit V1 were included in a multicentre, double-blind, randomised, placebo-controlled study (EudraCT: 2009-011826-32), that was performed to compare the efficacy of different doses of rifaximin vaginal tablets versus placebo for the treatment of BV. The patients underwent a randomisation visit and were distributed into 4 treatment groups: group A received 100 mg rifaximin vaginal tablet once daily for 5 days (n=10), group B received 25 mg rifaximin vaginal tablet once daily for 5 days (n=10), group C received 100 mg rifaximin vaginal tablet once daily for the first 2 days and placebo vaginal tablet for the remaining 3 days (n=9), group D received placebo vaginal tablet once daily for 5 days (n=10). Study medication was administered intra-vaginally at bedtime. After 7 to 10 days from the end of the therapy a follow-up visit (V3) was performed. Remission was evaluated at V3 according to Amsel's criteria (<3) and Gram stain Nugent score (≦3) (Table 1).

Standardized vaginal rinsings with 2 mL of saline were collected for analysis at V1 and V3 by flushing and re-aspirating the fluid through a 22 Gauge needle in the left, central and right upper vaginal vaults. The vaginal rinsings were subsequently stored at −80° C. until use.

b) MF10 Fractionation of Proteins

Prior to fractionation, pools H and BV containing 1 mg of protein each were prepared according to step a) and b) of Example 1. To constitute these pools, equal quantities of protein from each vaginal sample were mixed, dried down and resuspended in 280 μL of 90 mM Tris/10 mM EACA buffer pH 10.2 and urea 1 M. MF10 fractionation of proteins was performed using a 5-cartridge assembly with 5 kDa restriction membranes and 1 kDa, 5 kDa, 25 kDa, 50 kDa and 125 kDa separation membranes. Following chamber assembly, 100 mL of 90 mM Tris/10 mM EACA buffer pH 10.2 were added to the buffer reservoir and circulated around the electrodes. Protein pools (140 μL) were added to the chamber closest to the cathode for separate runs. Fractionations were performed at 250 V for 30 min. Following fraction collection, the lower fractions (1 to 5 kDa and 5 to 25 kDa) were desalted using Stage tips C18, 200 mL, according to manufacturer's instructions, and used for MS/MS analysis (pools HF and BV).

c) Mass Spectrometry Analysis

MS/MS analysis was carried out for H and BV pools and for the whole BV, A-R, A-N, B-R, B-N, C-R, C-N and D-N pools containing 50 μg of protein each. Each fraction or pool was resuspended in 50 μL of ammonium bicarbonate 50 mM pH 8. One μg/μL trypsin was added and the reaction was incubated at 37° C. overnight. After stopping the reaction by addiction of formic acid, the sample was vortexed and dried down. The pellet of the digested sample was resuspended in 10 μL of Buffer A (0.1% formic acid), and 0.2 μL of each sample in triplicate was run with blanks in between (Buffer A).

Digested peptides were separated by nano-LC using an Ultimate 3000 HPLC and autosampler system (Dionex, Amsterdam, The Netherlands). Samples (0.2 μL) were concentrated and desalted onto a micro C18 precolumn (500 μm×2 mm, Michrom Bioresources, Auburn, Calif.) with H₂O:CH₃CN (98:2, 0.05% trifluoroacetic acid, v/v) at 10 μL/min. After a 4-min wash, the precolumn was switched (Valco 10 port valve, Dionex) into line with a fritless C18 nano column (75 μm i.d.×10 cm containing 5 μm, 200 Å) manufactured according to Gatlin et al.

Peptides were eluted using a linear gradient of H₂O:CH₃CN (98:2, 0.1% formic acid, v/v) to H₂O:CH₃CN (64:36, 0.1% formic acid, v/v) at 250 nL/min over 30 min. High voltage (2000 V) was applied to low volume tee and the column tip positioned 0.5 cm from the heated capillary (T=280° C.) of an LTQ-Orbitrap Velos mass spectrometer. Positive ions were generated by electrospray and the Orbitrap operated in data-dependent acquisition mode (DDA). A survey scan of 350-1750 m/z was acquired (resolution=30000 at 400 m/z, with an accumulation target value of 1000000 ions). Up to the ten most abundant ions (>5000 counts) with charge states ≧2 were sequentially isolated and fragmented within the linear ion trap using collisionally induced dissociation with an activation q=0.25 and activation time of 30 ms at a target value of 30000 ions. Mass-to-charge ratios selected for MS/MS were dynamically excluded for 45 s.

Peak lists for MS/MS files from the LTQ-Orbitrap Velos were processed using Progenesis LC-MS v4. The software transforms the raw files of LC-MS runs into 2D profiles and aligns them to an arbitrarily chosen run using user-defined and automated vectors. The peptide intensities were normalized using proprietary code and used in the statistical analysis to calculate ANOVA and q-values and to deduce differentially expressed peptides among experimental pools (P<0.05). The Progenesis Stats package was used to perform a Principal Component Analysis (PCA) using the peptides with P<0.05. MS/MS spectra of differentiating peptides were searched against the Swiss-Prot database (version 15) using database search program MASCOT (Matrix Science, London, UK). Parent and fragment ions were searched with tolerances of ±4 ppm and ±0.5 Da, respectively. Peptide charge states were set at +2 and +3. ‘No enzyme’ was specified. Proteins and peptides were considered confidently identified when matches had a high ion score and were statistically significant (P<0.05) and (semi) tryptic. Following identification a filter was applied to select proteins of human origin and those produced by microorganisms associated to vaginal environment. Only proteins that exhibited ≧1.5-fold changes among experimental pools were considered. The results are reported in Table 7 and Table 8.

TABLE 7

| | Mascot search results | | | | |
|---|---|---|---|---|---|
| | SwissProt | Peptide | | | Ratio |
| Protein name | Acc. N. | count | Score | Anova (P) | BV/H |
| 10 kDa chaperonin | Q93G08 | 1 | 50.0 | 9.43 × 10⁻⁴ | |
| OS = Lactobacillus | | | | | |
| acidophilus | | | | | |
| 14-3-3 protein sigma | P31947 | 2 | 100.0 | 6.80 × 10⁻⁴ | 5.0 |
| 30S ribosomal protein S6 | Q5FN09 | 2 | 71.6 | 4.69 × 10⁻⁵ | −3.3 |
| OS = Lactobacillus | | | | | |
| acidophilus | | | | | |
| 50S ribosomal protein | Q045V5 | 1 | 86.9 | 2.71 × 10⁻⁴ | 3.8 |
| L7/L12 OS = Lactobacillus | | | | | |
| gasseri | | | | | |
| Acyl-CoA-binding protein | P07108 | 5 | 136.2 | 3.31 × 10⁻⁴ | 1.8 |
| Adipose most abundant | Q15847 | 1 | 36.6 | 2.73 × 10⁻⁵ | −20.2 |
| gene transcript 2 protein | | | | | |
| Alpha-1-acid glycoprotein 1 | P02763 | 5 | 182.3 | 1.42 × 10⁻⁴ | −7.9 |
| Alpha-1-acid glycoprotein 2 | P19652 | 2 | 103.3 | 1.57 × 10⁻⁴ | −28.6 |
| Alpha-1-antichymotrypsin | P01011 | 2 | 128.9 | 5.57 × 10⁻⁷ | 6.4 |
| Alpha-1-antitrypsin | P01009 | 15 | 650.5 | 4.30 × 10⁻³ | 1.5 |
| Alpha-1B-glycoprotein | P04217 | 2 | 95.7 | 1.30 × 10⁻³ | 1.9 |
| Alpha-2-HS-glycoprotein | P02765 | 7 | 238.2 | 4.64 × 10⁻⁶ | 5.0 |
| Alpha-2-macroglobulin | P01023 | 4 | 156.8 | 1.35 × 10⁻⁴ | 2.1 |
| Alpha-2-macroglobulin-like | A8K2U0 | 10 | 305.1 | 8.57 × 10⁻⁶ | 6.3 |
| protein 1 | | | | | |
| Annexin A1 | P04083 | 5 | 198.4 | 4.86 × 10⁻³ | −1.7 |
| Annexin A2 | P07355 | 7 | 254.4 | 2.02 × 10⁻⁷ | −7.1 |
| Annexin A3 | P12429 | 1 | 53.1 | 5.31 × 10⁻⁵ | 5.7 |
| Antithrombin-III | P01008 | 4 | 165.0 | 4.12 × 10⁻⁶ | 4.3 |
| Apolipoprotein A-I | P02647 | 7 | 309.2 | 2.84 × 10⁻⁵ | 2.7 |
| Apolipoprotein A-II | P02652 | 3 | 124.3 | 7.60 × 10⁻⁷ | −2.7 |
| Cadherin-1 | P12830 | 3 | 106.0 | 3.78 × 10⁻⁷ | 8.2 |
| Calcium-activated chloride | Q14CN2 | 1 | 23.1 | 1.74 × 10⁻⁴ | −5645.4 |
| channel regulator 4 | | | | | |
| Calmodulin-like protein 3 | P27482 | 3 | 83.4 | 8.55 × 10⁻¹⁰ | 29.2 |
| Calmodulin-like protein 5 | Q9NZT1 | 3 | 133.3 | 1.73 × 10⁻⁶ | 11.1 |
| Carbonic anhydrase 1 | P00915 | 8 | 318.3 | 5.20 × 10⁻⁵ | 2.9 |
| Catalase | P04040 | 1 | 68.5 | 1.98 × 10⁻² | −16.2 |
| Cathepsin B | P07858 | 3 | 124.4 | 7.41 × 10⁻⁵ | 4.0 |
| Ceruloplasmin | P00450 | 4 | 126.3 | 1.48 × 10⁻³ | 2.5 |
| Cold shock protein cspA | Q2YY16 | 1 | 51.1 | 5.09 × 10⁻⁵ | 48.0 |
| OS = Staphylococcus | | | | | |
| aureus | | | | | |
| Complement C3 | P01024 | 6 | 266.9 | 3.31 × 10⁻⁹ | 34.5 |
| Cornulin | Q9UBG3 | 36 | 1641.2 | 1.73 × 10⁻⁴ | −2.7 |
| Cystatin-A | P01040 | 6 | 300.9 | 3.31 × 10⁻⁶ | 3.6 |
| Cytidine deaminase | P32320 | 1 | 72.0 | 2.63 × 10⁻⁴ | 3.5 |
| Cytoplasmic tRNA 2- | Q6FLE5 | 3 | 109.4 | 2.12 × 10⁻⁶ | 2.7 |
| thiolation protein 2 | | | | | |
| OS = Candida glabrata | | | | | |
| Deleted in malignant brain | Q9UGM3 | 1 | 46.1 | 3.61 × 10⁻⁵ | 4.2 |
| tumors 1 protein | | | | | |
| Dermokine | Q6E0U4 | 3 | 113.8 | 5.90 × 10⁻⁴ | −4.0 |
| Desmocollin-2 | Q02487 | 7 | 257.9 | 2.10 × 10⁻⁵ | 8.9 |
| Desmoglein-3 | P32926 | 2 | 123.0 | 2.97 × 10⁻⁷ | 59.5 |
| Desmoplakin | P15924 | 5 | 169.8 | 1.15 × 10⁻⁵ | 4.2 |
| Enolase 1 | Q043Z5 | 2 | 94.9 | 1.82 × 10⁻⁵ | 2.6 |
| OS = Lactobacillus gasseri | | | | | |
| Enolase OS = Lactobacillus | Q5FKM6 | 5 | 216.0 | 1.51 × 10⁻⁵ | −17.8 |
| acidophilus | | | | | |
| Enolase OS = Lactobacillus | A8YUV4 | 6 | 350.9 | 5.81 × 10⁻³ | −10.0 |
| helveticus | | | | | |
| ERO1-like protein alpha | Q96HE7 | 2 | 107.4 | 5.42 × 10⁻⁷ | 90.2 |
| Fatty acid-binding protein, | Q01469 | 5 | 176.5 | 3.91 × 10⁻⁵ | −8.4 |
| epidermal | | | | | |
| Fibrinogen alpha chain | P02671 | 13 | 598.0 | 1.43 × 10⁻⁴ | 2.7 |
| Fibronectin | P02751 | 1 | 55.8 | 3.63 × 10⁻² | 6.0 |
| Filaggrin | P20930 | 28 | 1869.5 | 1.12 × 10⁻³ | −6.9 |
| Filaggrin-2 | Q5D862 | 2 | 111.7 | 3.65 × 10⁻⁴ | −6.0 |
| Flavin reductase | P30043 | 1 | 137.5 | 1.12 × 10⁻² | 2.4 |
| Galectin-3-binding protein | Q08380 | 1 | 57.9 | 3.01 × 10⁻⁵ | −8.8 |
| Glyceraldehyde-3- | P04406 | 7 | 318.6 | 8.60 × 10⁻⁵ | 13.5 |
| phosphate dehydrogenase | | | | | |
| Glycine cleavage system H | P23434 | 1 | 49.2 | 3.82 × 10⁻⁸ | 53.5 |
| protein, mitochondrial | | | | | |
| Haptoglobin | P00738 | 9 | 343.2 | 3.73 × 10⁻⁵ | 4.6 |
| Heme-binding protein 2 | Q9Y5Z4 | 3 | 118.9 | 2.54 × 10⁻⁵ | 2.8 |
| Hemoglobin subunit beta | P68871 | 41 | 2370.6 | 2.93 × 10⁻⁵ | 1.8 |
| Hemopexin | P02790 | 1 | 50.1 | 1.87 × 10⁻³ | −1.7 |
| Ig gamma-1 chain C region | P01857 | 9 | 473.8 | 1.41 × 10⁻⁶ | 8.0 |
| Ig gamma-2 chain C region | P01859 | 2 | 67.8 | 4.94 × 10⁻⁶ | 12.6 |
| Ig heavy chain V-III region | P01764 | 1 | 99.7 | 2.39 × 10⁻⁵ | 6.4 |
| VH26 | | | | | |
| Ig kappa chain C region | P01834 | 7 | 396.8 | 1.68 × 10⁻⁶ | 7.1 |
| Ig kappa chain V-I region | P01598 | 1 | 70.3 | 2.77 × 10⁻³ | 8.2 |
| EU | | | | | |
| Ig kappa chain V-II region | P01617 | 1 | 33.7 | 4.60 × 10⁻² | 3.5 |
| TEW | | | | | |
| Ig kappa chain V-III region | P01620 | 1 | 83.3 | 2.54 × 10⁻⁵ | 11.7 |
| SIE | | | | | |
| Ig kappa chain V-IV region | P06312 | 1 | 100.8 | 4.25 × 10⁻⁶ | 24.4 |
| Ig lambda chain V-III region | P80748 | 1 | 74.6 | 1.66 × 10⁻⁴ | 5.2 |
| LOI | | | | | |
| Ig lambda chain V-IV | P01717 | 1 | 40.0 | 1.54 × 10⁻² | 7.8 |
| region Hil | | | | | |
| Ig lambda-1 chain C | P0CG04 | 1 | 52.6 | 7.00 × 10⁻⁶ | 6.8 |
| regions | | | | | |
| Ig lambda-3 chain C | P0CG06 | 5 | 231.0 | 1.13 × 10⁻⁵ | 5.3 |
| regions | | | | | |
| Ig mu chain C region | P01871 | 1 | 33.8 | 2.92 × 10⁻² | −6.1 |
| IgGFc-binding protein | Q9Y6R7 | 2 | 57.3 | 2.80 × 10⁻⁵ | 12.8 |
| Immunoglobulin J chain | P01591 | 2 | 103.5 | 3.42 × 10⁻⁴ | 3.7 |
| Inter-alpha-trypsin inhibitor | P19827 | 1 | 44.8 | 1.91 × 10⁻⁵ | 36.9 |
| heavy chain H1 | | | | | |
| Interleukin-1 receptor | P18510 | 7 | 482.8 | 5.06 × 10⁻⁸ | 12.1 |
| antagonist protein | | | | | |
| Keratin, type I cytoskeletal | P13645 | 12 | 555.1 | 1.22 × 10⁻⁴ | 30.3 |
| 10 | | | | | |
| Keratin, type I cytoskeletal | P19012 | 2 | 61.5 | 4.42 × 10⁻⁵ | −8.2 |
| 15 | | | | | |
| Keratin, type I cytoskeletal | Q04695 | 3 | 130.2 | 1.02 × 10⁻² | −1.5 |
| 17 | | | | | |
| Keratin, type I cytoskeletal 9 | P35527 | 4 | 89.9 | 5.60 × 10⁻⁶ | 35.0 |
| Keratin, type II cytoskeletal 1 | P04264 | 23 | 1042.1 | 9.23 × 10⁻⁷ | 4.9 |
| Keratin, type II cytoskeletal | P35908 | 9 | 382.2 | 2.59 × 10⁻⁶ | 39.8 |
| 2 epidermal | | | | | |
| Keratin, type II cytoskeletal 5 | P13647 | 3 | 89.7 | 2.17 × 10⁻⁴ | 2.0 |
| Keratin, type II cytoskeletal | Q8N1N4 | 2 | 110.3 | 4.11 × 10⁻³ | −8.3 |
| 78 | | | | | |
| Lactotransferrin | P02788 | 8 | 432.9 | 1.82 × 10⁻⁴ | 1.5 |
| Ladinin-1 | O00515 | 1 | 55.9 | 5.57 × 10⁻³ | −8.1 |
| Leukocyte elastase | P30740 | 8 | 264.9 | 1.67 × 10⁻⁷ | 23.3 |
| inhibitor | | | | | |
| L-lactate dehydrogenase B | P07195 | 1 | 37.3 | 3.22 × 10⁻⁵ | 13.5 |
| chain | | | | | |
| L-lactate dehydrogenase | Q5HL31 | 1 | 29.9 | 1.27 × 10⁻² | 3.6 |
| OS = Staphylococcus | | | | | |
| epidermidis | | | | | |
| Lymphocyte-specific | P33241 | 2 | 56.8 | 2.38 × 10⁻⁷ | 21.4 |
| protein 1 | | | | | |
| Mucin-21 | Q5SSG8 | 1 | 119.9 | 6.37 × 10⁻³ | −5.9 |
| Mucin-5B | Q9HC84 | 3 | 117.2 | 4.66 × 10⁻⁴ | 3.7 |
| Myeloblastin | P24158 | 2 | 78.7 | 1.10 × 10⁻² | −5.1 |
| Myristoylated alanine-rich | P29966 | 5 | 190.7 | 1.01 × 10⁻³ | −2.4 |
| C-kinase substrate | | | | | |
| Netrin-G2 | Q96CW9 | 1 | 52.8 | 4.99 × 10⁻² | 3.5 |
| Neuroblast differentiation- | Q09666 | 13 | 549.6 | 8.92 × 10⁻⁵ | 1.7 |
| associated protein AHNAK | | | | | |
| Neutrophil gelatinase- | P80188 | 1 | 70.7 | 9.40 × 10⁻⁷ | 19.7 |
| associated lipocalin | | | | | |
| NSFL1 cofactor p47 | Q9UNZ2 | 2 | 76.9 | 1.90 × 10⁻⁸ | 521.1 |
| Olfactory receptor 5B2 | Q96R09 | 1 | 37.9 | 1.61 × 10⁻⁴ | 29.4 |
| Pericentriolar material 1 | Q15154 | 1 | 33.2 | 1.74 × 10⁻⁴ | −2.6 |
| protein | | | | | |
| Perilipin-3 | O60664 | 7 | 315.4 | 6.18 × 10⁻⁶ | 1.9 |
| Periplakin | O60437 | 12 | 547.6 | 3.01 × 10⁻³ | 3.5 |
| Phosphatidylethanolamine- | P30086 | 1 | 49.8 | 2.29 × 10⁻² | 5.2 |
| binding protein 1 | | | | | |
| Phosphocarrier protein HPr | Q9KJV3 | 1 | 80.6 | 1.92 × 10⁻³ | 1.7 |
| OS = Lactobacillus casei | | | | | |
| Plastin-2 | P13796 | 2 | 64.8 | 3.88 × 10⁻⁶ | 16.7 |
| Probable cation- | Q4VNC0 | 2 | 54.8 | 2.64 × 10⁻² | −3.5 |
| transporting ATPase 13A5 | | | | | |
| Protein FAM25 | B3EWG3 | 2 | 51.6 | 3.90 × 10⁻⁶ | −11.2 |
| Protein S100-A11 | P31949 | 1 | 53.7 | 1.45 × 10⁻⁴ | 3.3 |
| Protein S100-A14 | Q9HCY8 | 3 | 95.3 | 1.46 × 10⁻⁷ | 9.1 |
| Protein S100-A7 | P31151 | 3 | 140.1 | 1.15 × 10⁻⁵ | −4.3 |
| Protein S100-A9 | P06702 | 18 | 763.9 | 2.27 × 10⁻⁷ | −4.0 |
| Protein S100-P | P25815 | 2 | 110.8 | 6.76 × 10⁻³ | 1.7 |
| Protein-glutamine gamma- | Q08188 | 3 | 145.8 | 3.88 × 10⁻⁵ | 14.9 |
| glutamyltransferase E | | | | | |
| Pyruvate kinase isozymes | P14618 | 3 | 107.4 | 4.27 × 10⁻⁵ | 3.0 |
| M1/M2 | | | | | |
| Ras GTPase-activating-like | P46940 | 6 | 241.7 | 1.00 × 10⁻⁵ | 10.3 |
| protein IQGAP1 | | | | | |
| Repetin | Q6XPR3 | 7 | 267.4 | 1.13 × 10⁻⁵ | −9.2 |
| Resistin | Q9HD89 | 1 | 59.3 | 7.23 × 10⁻⁵ | 11.2 |
| Semenogelin-1 | P04279 | 3 | 159.6 | 1.54 × 10⁻⁸ | −12.9 |
| Serotransferrin | P02787 | 7 | 289.0 | 3.50 × 10⁻⁵ | 2.9 |
| Serpin B12 | Q96P63 | 2 | 111.7 | 5.81 × 10⁻⁵ | 6.6 |
| Serpin B13 | Q9UIV8 | 2 | 97.5 | 2.94 × 10⁻² | 4.4 |
| Serpin B4 | P48594 | 1 | 23.4 | 8.91 × 10⁻³ | −45.8 |
| Serum albumin | P02768 | 62 | 2895.3 | 1.18 × 10⁻⁶ | 3.7 |
| Small proline-rich protein 3 | Q9UBC9 | 4 | 131.7 | 2.29 × 10⁻⁴ | 2.0 |
| Superoxide dismutase [Cu—Zn] | P00441 | 2 | 68.9 | 4.89 × 10⁻³ | −49.2 |
| Suprabasin | Q6UWP8 | 4 | 153.8 | 1.58 × 10⁻⁵ | −17.5 |
| Synaptic vesicle membrane | Q99536 | 2 | 137.7 | 1.02 × 10⁻³ | 5.3 |
| protein VAT-1 homolog | | | | | |
| Thioredoxin domain- | Q9BRA2 | 1 | 44.6 | 2.53 × 10⁻⁶ | 43.6 |
| containing protein 17 | | | | | |
| Transaldolase | P37837 | 2 | 82.3 | 3.06 × 10⁻³ | 11.4 |
| Transcription factor PDR8 | Q06149 | 1 | 38.9 | 4.32 × 10⁻² | −36.2 |
| OS = Saccharomyces | | | | | |
| cerevisiae | | | | | |
| Transthyretin | P02766 | 5 | 194.3 | 2.12 × 10⁻⁵ | 2.0 |
| Triosephosphate | Q5FL49 | 6 | 275.6 | 3.47 × 10⁻³ | −7.7 |
| isomerase | | | | | |
| OS = Lactobacillus | | | | | |
| acidophilus | | | | | |
| Triosephosphate | A8YUE4 | 1 | 22.3 | 1.17 × 10⁻⁴ | 8.1 |
| isomerase | | | | | |
| OS = Lactobacillus | | | | | |
| helveticus | | | | | |
| Vitamin D-binding protein | P02774 | 2 | 80.6 | 2.08 × 10⁻² | −2.8 |

TABLE 8

| | | Mascot search results | | | | | | | | | |
|---|---|---|---|---|---|---|---|---|---|---|---|
| | SwissProt | Pept. | | Anova | Ratio X/H | | | | | | |
| Protein name^{a} | Acc. N. | count | Score | (P) | A-N | A-R | B-N | B-R | C-N | C-R | D-N |
| | | 7 | 374.2 | 7.31 × 10⁻¹² | −2.2 | 0.0 | −2.7 | 0.0 | −2.8 | 7.0 | 4.6 |
| 2,4-dienoyl-CoA | Q16698 | 4 | 168.0 | 1.01 × 10⁻⁵ | 0.0 | 0.0 | 0.0 | 0.0 | −2.2 | −2.3 | 0.0 |
| reductase, mitochondrial | | | | | | | | | | | |
| 30S ribosomal | Q5FM66 | 3 | 144.9 | 3.52 × 10⁻⁵ | 1.6 | 1.1 | −1.4 | −1.7 | 1.1 | −2.6 | 2.1 |
| protein S11 OS = | | | | | | | | | | | |
| Lactobacillus acidophilus | | | | | | | | | | | |
| 50S ribosomal | Q74L75 | 2 | 107.3 | 3.58 × 10⁻¹¹ | −1.7 | −1.4 | −1.5 | −2.5 | −2.9 | 1.6 | −3.1 |
| protein L6 OS = | | | | | | | | | | | |
| Lactobacillus johnsonii | | | | | | | | | | | |
| 60 kDa | Q045Q8 | 5 | 327.7 | 9.45 × 10⁻¹² | 1.3 | −1.6 | 1.1 | 3.0 | −1.9 | 1.0 | 2.2 |
| chaperonin OS = | | | | | | | | | | | |
| Lactobacillus gasseri | | | | | | | | | | | |
| 60 kDa | Q04E64 | 4 | 104.6 | 4.75 × 10⁻⁸ | −1.2 | 1.1 | 1.9 | 1.3 | 1.2 | −1.6 | −1.1 |
| chaperonin OS = | | | | | | | | | | | |
| Oenococcus oeni | | | | | | | | | | | |
| 60S ribosomal | P47914 | 1 | 57.6 | 3.84 × 10⁻⁶ | 14.7 | 27.6 | 8.1 | 21.6 | 7.1 | 13.3 | −11.5 |
| protein L29 | | | | | | | | | | | |
| Actin OS = | A5DQP9 | 10 | 393.7 | 1.03 × 10⁻⁹ | 1.4 | −1.7 | 1.2 | −1.1 | 1.3 | 1.8 | −1.0 |
| Pichia guilliermondii | | | | | | | | | | | |
| Acyl-CoA-binding | P07108 | 6 | 351.9 | 2.73 × 10⁻⁸ | 0.0 | 0.0 | 1.5 | 0.0 | 2.4 | 1.5 | 1.6 |
| protein | | | | | | | | | | | |
| ADP-ribosyl cyclase 2 | Q10588 | 2 | 89.0 | 2.95 × 10⁻⁹ | −2.4 | −2.8 | 0.0 | −3.0 | 0.0 | −1.8 | 2.1 |
| Afamin | P43652 | 3 | 97.7 | 2.21 × 10⁻⁶ | −2.3 | −1.6 | −1.6 | −1.6 | 0.0 | −3.3 | 0.0 |
| Alpha-1-acid | P02763 | 5 | 308.5 | 2.44 × 10⁻¹⁵ | 0.0 | −2.3 | 0.0 | −7.8 | 0.0 | 0.0 | 0.0 |
| glycoprotein 1 | | | | | | | | | | | |
| Alpha-1-acid | P19652 | 1 | 53.6 | 1.45 × 10⁻⁹ | 2.0 | −1.5 | 0.0 | −19.5 | 0.0 | 1.9 | 4.1 |
| glycoprotein 2 | | | | | | | | | | | |
| Alpha-1-antitrypsin | P01009 | 32 | 2091.6 | 1.43 × 10⁻¹⁴ | 0.0 | 0.0 | 0.0 | −3.3 | −1.6 | −1.8 | −2.7 |
| Alpha-1B- | P04217 | 6 | 274.1 | 6.66 × 10⁻¹⁵ | −1.7 | −4.2 | −2.4 | −14.9 | −2.9 | −2.6 | 0.0 |
| glycoprotein | | | | | | | | | | | |
| Alpha-2- | P08697 | 3 | 105.9 | 4.50 × 10⁻¹¹ | 1.6 | 3.2 | 1.7 | 3.9 | 1.9 | 2.0 | 1.8 |
| antiplasmin | | | | | | | | | | | |
| Alpha-2-HS- | P02765 | 2 | 90.4 | 4.81 × 10⁻⁸ | 2.8 | 0.0 | 1.5 | −1.7 | 1.6 | 1.8 | 0.0 |
| glycoprotein | | | | | | | | | | | |
| Alpha-2- | P01023 | 29 | 1867.5 | 3.13 × 10⁻⁹ | 0.0 | −2.0 | −2.5 | −10.6 | −3.1 | −1.9 | 0.0 |
| macroglobulin | | | | | | | | | | | |
| Alpha-2- | A8K2U0 | 66 | 3533.1 | 5.62 × 10⁻⁷ | −1.7 | −1.9 | −1.5 | 0.0 | −1.8 | −1.7 | 0.0 |
| macroglobulin-like | | | | | | | | | | | |
| protein 1 | | | | | | | | | | | |
| Alpha-actinin-4 | O43707 | 11 | 568.4 | 7.72 × 10⁻⁹ | 0.0 | −2.6 | −2.2 | −3.9 | −1.5 | 0.0 | 0.0 |
| Alpha-amylase 1 | P04745 | 4 | 120.3 | 3.34 × 10⁻¹⁰ | 2.6 | 1.9 | 1.5 | 1.8 | 0.0 | 0.0 | −6.8 |
| Alpha-enolase | P06733 | 15 | 721.6 | 2.95 × 10⁻⁸ | 0.0 | 0.0 | 0.0 | 0.0 | 0.0 | 0.0 | −1.7 |
| Angiotensinogen | P01019 | 7 | 377.7 | 1.31 × 10⁻¹³ | −1.8 | 0.0 | −1.5 | −5.5 | 0.0 | −1.9 | 0.0 |
| Annexin A1 | P04083 | 28 | 2268.8 | 5.63 × 10⁻⁴ | 0.0 | 2.5 | 3.8 | 0.0 | 2.4 | 0.0 | 8.1 |
| Annexin A11 | P50995 | 5 | 188.9 | 5.55 × 10⁻¹⁶ | −4.3 | −1.7 | 2.5 | −5.7 | 0.0 | −5.2 | 2.8 |
| Annexin A2 | P07355 | 31 | 2235.8 | 7.75 × 10⁻³ | 1.9 | 4.1 | 3.0 | 3.3 | 2.4 | 2.2 | 7.6 |
| Annexin A3 | P12429 | 20 | 1106.1 | 7.88 × 10⁻¹⁵ | −2.1 | 0.0 | 1.9 | −2.6 | 0.0 | −2.5 | 0.0 |
| Annexin A4 | P09525 | 6 | 429.4 | 9.77 × 10⁻⁷ | −2.4 | −2.5 | 0.0 | −3.7 | 0.0 | −3.3 | 1.6 |
| Annexin A6 | P08133 | 4 | 156.5 | 1.71 × 10⁻⁸ | −2.1 | −1.8 | 1.9 | −4.9 | 0.0 | −1.7 | 2.3 |
| Annexin A7 | P20073 | 1 | 55.1 | 1.72 × 10⁻⁶ | −1.8 | 0.0 | 0.0 | −2.2 | 0.0 | −3.3 | −9.2 |
| Antithrombin-III | P01008 | 7 | 334.9 | 9.81 × 10⁻¹⁰ | 1.5 | 2.0 | 0.0 | −1.9 | 0.0 | 0.0 | 1.7 |
| Apolipoprotein A-I | P02647 | 15 | 762.6 | 1.11 × 10⁻¹⁶ | 5.4 | 0.0 | −1.8 | −2.0 | −1.7 | 3.7 | 0.0 |
| Apolipoprotein A-IV | P06727 | 1 | 38.6 | 3.28 × 10⁻¹⁰ | 0.0 | −5.0 | 1.7 | −4.5 | −1.8 | −3.0 | −7.7 |
| Azurocidin | P20160 | 4 | 185.7 | 1.55 × 10⁻⁸ | −3.3 | −1.5 | 0.0 | 0.0 | −1.8 | −3.0 | 2.5 |
| Bactericidal/permeability- | P17213 | 8 | 366.6 | 5.56 × 10⁻⁸ | −1.9 | 0.0 | 0.0 | 0.0 | −1.6 | 0.0 | 1.6 |
| increasing protein | | | | | | | | | | | |
| Bactericidal/permeability- | Q8N4F0 | 6 | 362.6 | 6.32 × 10⁻⁹ | −7.9 | −19.2 | −4.8 | −4.3 | −4.6 | −6.1 | −1.5 |
| increasing protein-like 1 | | | | | | | | | | | |
| Cadherin-1 | P12830 | 3 | 212.3 | 2.20 × 10⁻¹⁴ | −2.9 | −5.2 | −1.9 | −20.1 | −3.1 | −6.1 | −2.5 |
| Calcium-activated | Q14CN2 | 7 | 263.0 | 7.29 × 10⁻¹¹ | −2.3 | −2.9 | 0.0 | 0.0 | −1.7 | −1.8 | 1.6 |
| chloride channel regulator 4 | | | | | | | | | | | |
| Calmodulin-like protein 3 | P27482 | 6 | 364.7 | 4.00 × 10⁻¹⁴ | 0.0 | −5.2 | 2.3 | −2.7 | −1.9 | 3.5 | 0.0 |
| Calmodulin-like protein 5 | Q9NZT1 | 5 | 274.7 | 1.71 × 10⁻⁷ | 0.0 | −1.7 | 0.0 | 0.0 | 0.0 | 0.0 | 0.0 |
| Calpain-1 catalytic subunit | P07384 | 9 | 330.3 | 1.09 × 10⁻¹¹ | −2.4 | −4.7 | −1.5 | −5.5 | −2.4 | −3.2 | 0.0 |
| Calpastatin | P20810 | 2 | 108.7 | 3.34 × 10⁻¹¹ | 4.4 | 7.3 | 1.9 | 4.5 | 2.9 | 3.8 | 0.0 |
| Carbonic anhydrase 1 | P00915 | 9 | 560.5 | 1.44 × 10⁻¹⁵ | −1.9 | −19.0 | −7.5 | −15.4 | −9.5 | 0.0 | −11.5 |
| Carcinoembryonic | P06731 | 9 | 527.8 | 1.74 × 10⁻¹³ | −2.2 | 0.0 | 0.0 | 0.0 | 0.0 | −2.2 | −1.8 |
| antigen-related cell | | | | | | | | | | | |
| adhesion molecule 5 | | | | | | | | | | | |
| Carcinoembryonic | P31997 | 2 | 170.3 | 1.09 × 10⁻⁹ | −3.5 | 0.0 | 0.0 | 0.0 | −1.5 | −3.2 | −22.4 |
| antigen-related cell | | | | | | | | | | | |
| adhesion molecule 8 | | | | | | | | | | | |
| Catalase | P04040 | 23 | 1408.1 | 0.00 | 0.0 | 0.0 | 2.4 | −4.0 | 0.0 | −2.3 | 0.0 |
| Cathepsin B | P07858 | 7 | 341.3 | 1.45 × 10⁻⁹ | 0.0 | 0.0 | 0.0 | 0.0 | 0.0 | 0.0 | 1.7 |
| Cathepsin D | P07339 | 3 | 154.2 | 4.78 × 10⁻⁷ | 0.0 | 1.5 | 2.9 | 1.7 | 0.0 | 0.0 | 0.0 |
| Cathepsin G | P08311 | 6 | 238.3 | 6.92 × 10⁻⁸ | 0.0 | 0.0 | 0.0 | 2.2 | 0.0 | 0.0 | 2.8 |
| Cellular retinoic | P29373 | 7 | 276.4 | 2.20 × 10⁻¹² | −2.6 | −2.9 | −1.7 | −4.7 | −1.6 | 2.0 | 2.2 |
| acid-binding protein 2 | | | | | | | | | | | |
| Ceruloplasmin | P00450 | 7 | 256.6 | 4.14 × 10⁻⁹ | 0.0 | −2.7 | −1.8 | −3.6 | −1.8 | 0.0 | −3.4 |
| Chloride intracellular | O00299 | 1 | 49.9 | 1.09 × 10⁻⁹ | 1.6 | 0.0 | −4.2 | −1.9 | −2.4 | 1.6 | −7.1 |
| channel protein 1 | | | | | | | | | | | |
| Cofilin-1 | P23528 | 3 | 191.8 | 6.85 × 10⁻⁷ | −1.5 | −4.0 | −4.5 | −3.1 | 0.0 | 2.6 | 3.8 |
| Complement C3 | P01024 | 57 | 3268.9 | 1.11 × 10⁻¹⁶ | 0.0 | −4.0 | −1.8 | −6.1 | −2.4 | −2.3 | −2.2 |
| Complement C4-A | P0C0L4 | 16 | 687.3 | 7.43 × 10⁻⁹ | 0.0 | −1.8 | 0.0 | −2.5 | 0.0 | 0.0 | 0.0 |
| Complement factor H | P08603 | 5 | 193.3 | 6.22 × 10⁻⁷ | 0.0 | 0.0 | −1.5 | 0.0 | 0.0 | 0.0 | 4.7 |
| Cornifin-B | P22528 | 2 | 67.7 | 3.25 × 10⁻¹³ | 1.6 | 1.5 | 0.0 | 2.8 | 1.8 | 2.1 | −3.5 |
| Cornulin | Q9UBG3 | 93 | 6695.0 | 3.06 × 10⁻² | 0.0 | 0.0 | 0.0 | 1.6 | −1.5 | 0.0 | 0.0 |
| Cystatin-A | P01040 | 12 | 823.0 | 7.00 × 10⁻⁸ | −2.7 | 0.0 | 0.0 | 0.0 | 2.8 | −1.7 | −2.1 |
| Cysteine-rich | P54108 | 6 | 275.7 | 1.82 × 10⁻⁹ | 0.0 | 3.9 | 0.0 | 7.3 | 1.6 | 1.6 | −15.2 |
| secretory protein 3 | | | | | | | | | | | |
| Cytidine deaminase | P32320 | 4 | 189.3 | 1.28 × 10⁻⁵ | 0.0 | 0.0 | 1.5 | 0.0 | 0.0 | 0.0 | 0.0 |
| D-dopachrome | P30046 | 1 | 40.1 | 9.36 × 10⁻⁸ | −2.1 | −4.4 | −1.5 | −6.0 | 0.0 | 0.0 | −1.6 |
| decarboxylase | | | | | | | | | | | |
| Delta(3,5)-Delta(2,4)- | Q13011 | 2 | 94.0 | 1.40 × 10⁻¹⁰ | −2.2 | −2.3 | −2.5 | −6.8 | 0.0 | −6.6 | −5.3 |
| dienoyl-CoA isomerase, | | | | | | | | | | | |
| mitochondrial | | | | | | | | | | | |
| Desmocollin-2 | Q02487 | 12 | 672.2 | 4.80 × 10⁻⁹ | 0.0 | 1.5 | 0.0 | 2.0 | 1.5 | 0.0 | −1.5 |
| Desmoglein-1 | Q02413 | 7 | 358.3 | 8.29 × 10⁻⁸ | −2.1 | −1.6 | 0.0 | 0.0 | −1.6 | −2.1 | −3.7 |
| Desmoglein-3 | P32926 | 9 | 369.9 | 7.73 × 10⁻⁸ | 0.0 | −1.9 | 0.0 | −4.2 | −1.5 | −1.7 | −2.1 |
| Desmoplakin | P15924 | 6 | 187.7 | 7.73 × 10⁻¹⁰ | −2.1 | −2.4 | 0.0 | −2.1 | 0.0 | −1.5 | −2.0 |
| Dipeptidyl peptidase 1 | P53634 | 4 | 147.0 | 1.13 × 10⁻⁸ | 3.5 | 2.1 | 2.7 | 0.0 | 1.9 | 2.8 | −1.7 |
| Elongation factor G | A8YXK3 | 4 | 113.1 | 4.50 × 10⁻⁶ | 1.6 | −1.1 | 1.6 | −1.1 | 2.3 | −1.3 | 2.2 |
| OS = Lactobacillus helveticus | | | | | | | | | | | |
| Elongation factor Ts | B7GQR9 | 4 | 198.6 | 5.21 × 10⁻¹¹ | −1.8 | 1.7 | 4.2 | −1.3 | 1.9 | 1.2 | 1.9 |
| OS = Bifidobacterium longum | | | | | | | | | | | |
| subsp. infantis | | | | | | | | | | | |
| Enolase 1 OS = | Q043Z5 | 8 | 528.4 | 2.94 × 10⁻¹¹ | −2.5 | −3.5 | −3.1 | −1.9 | −2.1 | 1.1 | 1.3 |
| Lactobacillus gasseri | | | | | | | | | | | |
| Enolase OS = | Q5FKM6 | 1 | 26.8 | 6.65 × 10⁻⁸ | −1.8 | −2.6 | −2.3 | −1.2 | −4.0 | −1.0 | 1.0 |
| Lactobacillus acidophilus | | | | | | | | | | | |
| Enolase OS = | Q1G9S9 | 1 | 21.1 | 1.23 × 10⁻¹⁰ | −2.7 | −2.7 | −2.7 | −3.0 | 1.1 | −3.9 | 0.0 |
| Lactobacillus delbrueckii | | | | | | | | | | | |
| subsp. bulgaricus | | | | | | | | | | | |
| Enolase OS = | A8YUV4 | 1 | 32.2 | 4.91 × 10⁻¹² | 1.9 | −1.6 | 1.6 | −1.2 | 1.9 | 1.6 | 1.3 |
| Lactobacillus helveticus | | | | | | | | | | | |
| Envoplakin | Q92817 | 4 | 159.3 | 1.96 × 10⁻¹¹ | 2.0 | 1.9 | 3.2 | 0.0 | 0.0 | 2.1 | 1.8 |
| ER membrane protein | P25574 | 3 | 87.7 | 1.84 × 10⁻¹³ | 1.5 | 2.5 | 1.6 | 2.9 | −1.1 | 1.6 | −1.1 |
| complex subunit 1 OS = | | | | | | | | | | | |
| Saccharomyces cerevisiae | | | | | | | | | | | |
| ERO1-like protein alpha | Q96HE7 | 7 | 343.3 | 1.22 × 10⁻⁷ | −1.7 | −1.7 | −1.5 | −2.1 | 0.0 | 1.7 | −1.8 |
| Extracellular matrix | Q16610 | 5 | 243.6 | 1.65 × 10⁻¹³ | 2.0 | 0.0 | 0.0 | 0.0 | 0.0 | 4.9 | 2.7 |
| protein 1 | | | | | | | | | | | |
| Ezrin | P15311 | 5 | 238.1 | 1.51 × 10⁻¹⁰ | −1.9 | −6.0 | −2.1 | −5.2 | −3.2 | 0.0 | 4.0 |
| F-actin-capping protein | P52907 | 6 | 312.2 | 1.99 × 10⁻¹⁴ | −1.7 | −6.5 | −1.8 | −8.5 | −2.4 | −2.5 | −3.3 |
| subunit alpha-1 | | | | | | | | | | | |
| Fatty acid-binding | Q01469 | 14 | 612.7 | 1.89 × 10⁻¹⁵ | 0.0 | 2.9 | 2.0 | 3.4 | 0.0 | 0.0 | 0.0 |
| protein, epidermal | | | | | | | | | | | |
| Fibrinogen alpha chain | P02671 | 10 | 464.2 | 2.18 × 10⁻¹² | 1.5 | −2.1 | −1.7 | −3.8 | −2.4 | 0.0 | −5.5 |
| Fibrinogen beta chain | P02675 | 14 | 746.4 | 0.00 | 0.0 | −4.6 | −2.2 | −7.6 | −2.6 | 0.0 | 0.0 |
| Fibrinogen gamma | P02679 | 8 | 570.5 | 1.86 × 10⁻¹² | −1.7 | −4.5 | −3.1 | −4.0 | −4.3 | −1.6 | −2.3 |
| chain | | | | | | | | | | | |
| Fibronectin | P02751 | 4 | 173.4 | 2.17 × 10⁻⁶ | −1.7 | −1.9 | 0.0 | −1.9 | −2.1 | −2.0 | −1.8 |
| Fibulin-1 | P23142 | 1 | 59.2 | 5.19 × 10⁻¹¹ | 5.5 | −2.5 | 0.0 | −2.2 | 0.0 | 1.5 | 1.8 |
| Filaggrin | P20930 | 48 | 2677.8 | 2.47 × 10⁻¹⁰ | 1.8 | 7.5 | 1.6 | 17.9 | 4.4 | 2.2 | −2.9 |
| Filamin-A | P21333 | 17 | 801.9 | 3.53 × 10⁻¹⁰ | −2.1 | −3.4 | 0.0 | −3.5 | −1.9 | −3.8 | −2.4 |
| Filamin-B | O75369 | 14 | 568.7 | 5.45 × 10⁻¹⁰ | −2.5 | −1.7 | 0.0 | 0.0 | −1.5 | −2.0 | 0.0 |
| Fructose-1,6- | P09467 | 1 | 48.6 | 3.98 × 10⁻¹² | −4.9 | −1.9 | −4.7 | −14.4 | −3.5 | −8.0 | 2.7 |
| bisphosphatase | | | | | | | | | | | |
| Fructose-bisphosphate | P04075 | 21 | 1144.6 | 1.89 × 10⁻¹⁵ | 0.0 | −4.4 | 0.0 | −4.2 | −2.0 | 0.0 | −1.6 |
| aldolase A | | | | | | | | | | | |
| Galectin-3-binding | Q08380 | 8 | 328.8 | 1.15 × 10⁻¹⁰ | −1.6 | −3.6 | −2.7 | −2.7 | 0.0 | 0.0 | 0.0 |
| protein | | | | | | | | | | | |
| Galectin-7 | P47929 | 2 | 92.5 | 1.44 × 10⁻¹⁰ | 0.0 | 0.0 | 0.0 | 0.0 | 0.0 | −1.9 | 5.2 |
| Gamma-glutamyl | Q92820 | 2 | 82.1 | 7.43 × 10⁻⁵ | 0.0 | 0.0 | 2.7 | 0.0 | 0.0 | 0.0 | 2.8 |
| hydrolase | | | | | | | | | | | |
| Gamma- | O75223 | 6 | 323.2 | 4.63 × 10⁻¹³ | 0.0 | 1.6 | 0.0 | 2.0 | 0.0 | 0.0 | −1.5 |
| glutamylcyclotransferase | | | | | | | | | | | |
| Gelsolin | P06396 | 15 | 854.6 | 2.97 × 10⁻⁸ | 0.0 | −1.6 | 0.0 | 0.0 | 0.0 | −1.5 | 3.0 |
| Glucosamine 6- | Q96EK6 | 1 | 60.7 | 1.35 × 10⁻³ | 2.4 | 8.6 | 3.6 | 13.0 | 5.7 | 4.3 | −10.3 |
| phosphate N- | | | | | | | | | | | |
| acetyltransferase | | | | | | | | | | | |
| Glucose-6-phosphate | P11413 | 5 | 172.0 | 1.18 × 10⁻¹⁴ | −2.4 | −11.2 | 1.6 | −9.0 | 0.0 | −4.5 | 2.9 |
| 1-dehydrogenase | | | | | | | | | | | |
| Glutaredoxin-1 | P35754 | 2 | 167.4 | 1.97 × 10⁻¹³ | −1.8 | −1.6 | 2.8 | −1.9 | 0.0 | −3.2 | −1.5 |
| Glutathione | P09211 | 9 | 648.4 | 1.47 × 10⁻¹³ | −3.0 | −6.1 | −2.4 | −2.9 | −2.3 | 0.0 | −2.6 |
| S-transferase P | | | | | | | | | | | |
| Glyceraldehyde-3- | P04406 | 12 | 805.8 | 4.33 × 10⁻¹⁵ | 2.1 | 0.0 | 0.0 | 0.0 | 0.0 | 5.0 | 0.0 |
| phosphate dehydrogenase | | | | | | | | | | | |
| Glyceraldehyde-3- | O32755 | 7 | 345.8 | 6.49 × 10⁻⁹ | −1.1 | 1.6 | −1.7 | 2.0 | −1.8 | −1.9 | −1.1 |
| phosphate dehydrogenase OS = | | | | | | | | | | | |
| Lactobacillus delbrueckii | | | | | | | | | | | |
| subsp. bulgaricus | | | | | | | | | | | |
| Glycodelin | P09466 | 5 | 412.7 | 1.11 × 10⁻¹⁶ | 13.5 | −5.9 | −1.9 | 0.0 | −3.2 | −2.1 | 2.0 |
| Haptoglobin | P00738 | 18 | 821.0 | 0.00 | 0.0 | −4.9 | −2.0 | −7.5 | −6.4 | 0.0 | −2.6 |
| Heat shock 70 kDa | P17066 | 10 | 410.2 | 3.02 × 10⁻¹⁰ | 0.0 | −2.1 | −2.6 | −1.6 | −1.7 | 0.0 | 3.8 |
| protein 6 | | | | | | | | | | | |
| Heat shock protein | P04792 | 15 | 865.1 | 6.11 × 10⁻¹⁵ | 2.3 | 7.3 | 0.0 | 8.9 | 1.8 | 2.2 | 0.0 |
| beta-1 | | | | | | | | | | | |
| Heme-binding | Q9Y5Z4 | 5 | 291.6 | 6.61 × 10⁻⁷ | 0.0 | −3.2 | 0.0 | −1.5 | 0.0 | 0.0 | −2.5 |
| protein 2 | | | | | | | | | | | |
| Hemoglobin subunit | P69905 | 17 | 916.8 | 0.00 | 0.0 | −22.3 | −14.8 | −33.4 | −16.1 | 1.6 | −4.9 |
| alpha | | | | | | | | | | | |
| Hemoglobin subunit | P68871 | 28 | 1846.5 | 0.00 | −1.9 | −28.9 | −18.9 | −41.7 | −16.8 | 0.0 | −9.1 |
| beta | | | | | | | | | | | |
| Hemoglobin subunit | P02042 | 3 | 174.5 | 2.22 × 10⁻¹⁶ | −2.1 | −15.7 | −6.2 | −15.2 | −8.5 | 0.0 | −15.4 |
| delta | | | | | | | | | | | |
| Hemoglobin subunit | P02008 | 1 | 27.6 | 3.01 × 10⁻⁹ | 2.2 | 1.7 | 2.7 | 1.8 | 3.4 | 2.2 | −2.5 |
| zeta | | | | | | | | | | | |
| Hemopexin | P02790 | 10 | 481.1 | 3.49 × 10⁻⁹ | −1.5 | −1.9 | 0.0 | −9.0 | −1.7 | −1.7 | 1.6 |
| Histidine-rich glycoprotein | P04196 | 4 | 164.2 | 1.89 × 10⁻⁶ | 0.0 | −1.8 | 0.0 | 0.0 | 1.5 | 1.6 | 31.4 |
| Histone H1.0 | P07305 | 3 | 189.1 | 1.05 × 10⁻¹² | 0.0 | 0.0 | 1.9 | 1.6 | 0.0 | 0.0 | −7.6 |
| Histone H1.3 | P16402 | 5 | 201.9 | 2.43 × 10⁻¹² | 0.0 | 2.4 | 1.5 | 3.0 | 2.7 | 0.0 | −2.7 |
| Ig alpha-1 chain C region | P01876 | 14 | 607.0 | 4.76 × 10⁻¹⁴ | 0.0 | 0.0 | 0.0 | 0.0 | 0.0 | 0.0 | −1.9 |
| Ig alpha-2 chain C regio | P01877 | 1 | 72.7 | 1.31 × 10⁻³ | 0.0 | −2.0 | 0.0 | −1.6 | −1.7 | −1.5 | −1.6 |
| Ig gamma-1 chain C region | P01857 | 34 | 2042.1 | 7.97 × 10⁻¹⁰ | 0.0 | 0.0 | 0.0 | 0.0 | 0.0 | 0.0 | −2.5 |
| Ig gamma-2 chain C region | P01859 | 10 | 543.1 | 1.60 × 10⁻⁸ | 0.0 | 0.0 | 0.0 | −3.4 | 0.0 | −1.5 | −3.4 |
| Ig gamma-4 chain C region | P01861 | 1 | 98.1 | 1.22 × 10⁻⁴ | 0.0 | 0.0 | 0.0 | 0.0 | 0.0 | −1.8 | −1.5 |
| Ig heavy chain V-III region BUT | P01767 | 2 | 84.9 | 3.38 × 10⁻⁹ | 0.0 | 0.0 | 0.0 | −1.6 | 0.0 | 0.0 | 2.8 |
| Ig heavy chain V-III region CAM | P01768 | 1 | 61.4 | 4.69 × 10⁻⁶ | 1.9 | 6.0 | 3.1 | 2.4 | 2.2 | 3.0 | −5.8 |
| Ig heavy chain V-III region GAL | P01781 | 3 | 117.6 | 6.74 × 10⁻⁹ | −2.9 | −4.9 | −4.4 | −7.5 | −6.3 | −5.1 | 0.0 |
| Ig heavy chain V-III region JON | P01780 | 1 | 48.6 | 7.61 × 10⁻⁴ | 0.0 | 0.0 | 0.0 | 0.0 | 0.0 | 0.0 | −39.6 |
| Ig heavy chain V-III region TEI | P01777 | 3 | 276.4 | 1.47 × 10⁻⁹ | 0.0 | 0.0 | 0.0 | 0.0 | 0.0 | 0.0 | −4.7 |
| Ig heavy chain V-III region VH26 | P01764 | 5 | 302.6 | 4.66 × 10⁻³ | 0.0 | 0.0 | 0.0 | −1.5 | 0.0 | 0.0 | 0.0 |
| Ig kappa chain C region | P01834 | 14 | 985.3 | 8.13 × 10⁻⁸ | −1.6 | 0.0 | 0.0 | 0.0 | −1.6 | −2.5 | −4.5 |
| Ig kappa chain V-I region AG | P01593 | 1 | 81.7 | 3.69 × 10⁻¹⁰ | 0.0 | −1.8 | 1.9 | −1.9 | −1.5 | −3.5 | −12.7 |
| Ig kappa chain V-I region BAN | P04430 | 1 | 105.2 | 3.81 × 10⁻³ | 0.0 | 0.0 | 0.0 | −1.5 | 0.0 | −2.1 | −41.7 |
| Ig kappa chain V-I region CAR | P01596 | 2 | 61.2 | 1.06 × 10⁻⁸ | −4.0 | −4.2 | −2.6 | −2.9 | −1.5 | −6.4 | 4.7 |
| Ig kappa chain V-I region DEE | P01597 | 6 | 346.1 | 7.10 × 10⁻¹¹ | 0.0 | 0.0 | 0.0 | −1.7 | 0.0 | −2.0 | −2.9 |
| Ig kappa chain V-I region | P01602 | 5 | 338.6 | 6.92 × 10⁻¹⁴ | 0.0 | 0.0 | 0.0 | 0.0 | 0.0 | −1.5 | −5.5 |
| HK102 (Fragment) | | | | | | | | | | | |
| Ig kappa chain V-I region Lay | P01605 | 1 | 67.8 | 2.22 × 10⁻¹⁶ | 1.7 | 0.0 | 1.7 | 0.0 | 0.0 | −1.5 | −17.8 |
| Ig kappa chain V-I region Mev | P01612 | 1 | 84.8 | 3.46 × 10⁻¹⁰ | 0.0 | 0.0 | 1.9 | 0.0 | 1.7 | −1.6 | −91.9 |
| Ig kappa chain V-I region WEA | P01610 | 1 | 20.3 | 9.58 × 10⁻⁶ | 3.2 | 0.0 | 2.4 | 4.5 | 0.0 | 2.2 | −2.9 |
| Ig kappa chain V-I region Wes | P01611 | 1 | 25.9 | 7.69 × 10⁻¹¹ | −2.4 | −5.6 | 1.7 | −2.9 | −2.0 | −4.5 | 0.0 |
| Ig kappa chain V-II region | P06309 | 4 | 185.8 | 4.83 × 10⁻⁸ | 0.0 | −1.8 | 0.0 | −2.1 | 0.0 | 0.0 | 0.0 |
| GM607 (Fragment) | | | | | | | | | | | |
| Ig kappa chain V-III region B6 | P01619 | 1 | 71.5 | 1.73 × 10⁻⁶ | 0.0 | −1.7 | 0.0 | −1.5 | −2.1 | −1.9 | −5.9 |
| Ig kappa chain V-III region CL | P04207 | 3 | 240.2 | 1.30 × 10⁻⁹ | 0.0 | 0.0 | 0.0 | 0.0 | 0.0 | 0.0 | −6.2 |
| Ig kappa chain V-III region HAH | P18135 | 9 | 469.9 | 4.71 × 10⁻⁸ | 0.0 | 0.0 | 0.0 | −1.5 | 0.0 | −2.1 | −4.6 |
| Ig kappa chain V-III region POM | P01624 | 1 | 65.4 | 4.84 × 10⁻⁴ | 0.0 | 0.0 | −2.6 | −2.1 | −1.7 | −3.3 | −83.6 |
| Ig kappa chain V-III region | P04433 | 3 | 210.4 | 5.79 × 10⁻³ | 0.0 | −1.5 | 0.0 | −1.6 | 0.0 | −2.3 | −2.0 |
| VG (Fragment) | | | | | | | | | | | |
| Ig kappa chain V-IV region | P06312 | 5 | 392.0 | 6.11 × 10⁻⁸ | 0.0 | 0.0 | 0.0 | 0.0 | 0.0 | −2.1 | −2.7 |
| (Fragment) | | | | | | | | | | | |
| Ig kappa chain V-IV region | P83593 | 1 | 44.9 | 1.79 × 10⁻⁵ | 2.0 | 0.0 | 2.1 | 2.3 | 0.0 | −1.7 | −6.4 |
| STH (Fragment) | | | | | | | | | | | |
| Ig lambda chain V-I region HA | P01700 | 2 | 112.4 | 1.66 × 10⁻⁸ | 0.0 | 0.0 | 0.0 | 0.0 | 0.0 | 0.0 | −3.6 |
| Ig lambda chain V-III region LOI | P80748 | 3 | 163.2 | 2.39 × 10⁻¹³ | 0.0 | −1.5 | 0.0 | 0.0 | 1.5 | −1.5 | −3.2 |
| Ig lambda chain V-III region SH | P01714 | 2 | 190.98 | 7.28 × 10⁻¹⁰ | 0.0 | 0.0 | 0.0 | 0.0 | 0.0 | −1.8 | −8.0 |
| Ig lambda chain V-IV region Hil | P01717 | 2 | 118.3 | 2.34 × 10⁻⁶ | 0.0 | −1.8 | 0.0 | −2.7 | 0.0 | −2.4 | −3.8 |
| Ig lambda chain V-VI region EB4 | P06319 | 2 | 121.6 | 7.25 × 10⁻⁴ | 0.0 | 0.0 | 0.0 | −2.0 | 0.0 | 0.0 | −2.1 |
| Ig lambda-1 chain C regions | P0CG04 | 2 | 146.0 | 2.33 × 10⁻⁸ | 0.0 | −1.8 | 0.0 | −1.5 | 0.0 | −2.6 | −11.4 |
| Ig lambda-2 chain C regions | P0CG05 | 11 | 661.9 | 3.61 × 10⁻⁷ | 0.0 | −1.7 | 0.0 | −1.9 | 0.0 | −2.2 | −3.7 |
| Ig lambda-7 chain C region | A0M8Q6 | 1 | 37.4 | 5.87 × 10⁻¹³ | 2.9 | −2.0 | 1.9 | 4.5 | 0.0 | 3.3 | −957.1 |
| Ig mu chain C region | P01871 | 9 | 440.2 | 1.11 × 10⁻¹⁶ | 0.0 | −1.8 | −1.9 | −8.1 | −2.8 | 0.0 | −1.5 |
| IgGFc-binding protein | Q9Y6R7 | 24 | 1123.5 | 2.22 × 10⁻⁹ | 0.0 | 0.0 | 0.0 | 0.0 | 0.0 | 0.0 | −1.6 |
| Immunoglobulin J chain | P01591 | 3 | 115.0 | 2.40 × 10⁻¹⁰ | 3.3 | 1.5 | 2.2 | 4.1 | 2.3 | 2.2 | −50.8 |
| Insulin-like growth factor- | Q16270 | 2 | 120.7 | 7.84 × 10⁻³ | 16.3 | 1.7 | 2.8 | 27.6 | 4.8 | 7.7 | 0.0 |
| binding protein 7 | | | | | | | | | | | |
| Inter-alpha-trypsin inhibitor | P19827 | 2 | 141.2 | 6.65 × 10⁻⁵ | −2.1 | −2.1 | −2.3 | −2.2 | 0.0 | 0.0 | 0.0 |
| heavy chain H1 | | | | | | | | | | | |
| Inter-alpha-trypsin inhibitor | P19823 | 3 | 137.4 | 4.59 × 10⁻⁸ | 3.9 | −2.4 | −1.9 | −1.5 | 0.0 | 6.2 | 0.0 |
| heavy chain H2 | | | | | | | | | | | |
| Inter-alpha-trypsin inhibitor | Q14624 | 2 | 57.2 | 5.98 × 10⁻¹¹ | 0.0 | −2.3 | 0.0 | −2.6 | 0.0 | 0.0 | −12.0 |
| heavy chain H4 | | | | | | | | | | | |
| Interleukin-1 family member 9 | Q9NZH8 | 2 | 77.6 | 2.66 × 10⁻⁵ | 0.0 | −2.0 | 2.3 | 0.0 | 0.0 | −1.5 | 0.0 |
| Interleukin-1 receptor | P18510 | 10 | 598.9 | 3.64 × 10⁻¹⁰ | −1.5 | −2.1 | 1.5 | −2.3 | 0.0 | −2.6 | −1.7 |
| antagonist protein | | | | | | | | | | | |
| Involucrin | P07476 | 20 | 1063.2 | 4.19 × 10⁻¹³ | 2.5 | 5.3 | 0.0 | 4.3 | 0.0 | 4.7 | 0.0 |
| Isocitrate dehydrogenase | O75874 | 2 | 77.3 | 1.28 × 10⁻¹⁰ | −3.6 | −4.0 | −3.4 | −44.2 | 0.0 | −5.6 | −4.0 |
| [NADP] cytoplasmic | | | | | | | | | | | |
| Kallikrein-10 | O43240 | 4 | 175.2 | 9.09 × 10⁻⁷ | −2.9 | −1.5 | −2.6 | 0.0 | −1.9 | −2.4 | 5.4 |
| Kallikrein-11 | Q9UBX7 | 5 | 214.2 | 1.43 × 10⁻¹⁰ | −3.3 | −5.4 | −3.9 | −3.7 | 0.0 | −1.5 | −2.8 |
| Kallikrein-12 | Q9UKR0 | 1 | 64.7 | 7.93 × 10⁻⁵ | 2.3 | 5.6 | 2.0 | 1.8 | 1.5 | 1.5 | 2.0 |
| Kallikrein-13 | Q9UKR3 | 5 | 225.2 | 2.67 × 10⁻⁹ | 0.0 | 0.0 | 0.0 | 1.6 | 0.0 | 0.0 | 0.0 |
| Kallikrein-6 | Q92876 | 3 | 128.1 | 5.21 × 10⁻⁹ | −2.0 | −3.1 | −1.5 | 0.0 | −2.7 | −3.5 | 0.0 |
| Keratin, type I cytoskeletal 10 | P13645 | 4 | 293.7 | 2.82 × 10⁻¹² | 0.0 | 2.8 | 0.0 | 3.5 | 1.7 | 0.0 | 0.0 |
| Keratin, type I cytoskeletal 13 | P13646 | 38 | 2352.6 | 3.33 × 10⁻¹⁶ | 0.0 | 3.3 | 2.4 | 4.1 | 1.6 | 0.0 | 0.0 |
| Keratin, type I cytoskeletal 14 | P02533 | 4 | 141.7 | 4.91 × 10⁻¹¹ | 0.0 | −2.5 | 0.0 | −2.3 | −2.0 | 0.0 | −1.5 |
| Keratin, type I cytoskeletal 15 | P19012 | 1 | 24.1 | 2.14 × 10⁻⁶ | 1.9 | 3.3 | 1.7 | 4.1 | 3.0 | 2.1 | 2.5 |
| Keratin, type I cytoskeletal 16 | P08779 | 3 | 97.8 | 2.44 × 10⁻¹⁵ | 0.0 | 1.8 | 0.0 | 0.0 | 2.2 | 0.0 | −13.5 |
| Keratin, type I cytoskeletal 19 | P08727 | 1 | 75.8 | 3.78 × 10⁻⁴ | −2.0 | 1.7 | 0.0 | 1.5 | −1.5 | 0.0 | 1.6 |
| Keratin, type I cytoskeletal 9 | P35527 | 2 | 80.6 | 8.01 × 10⁻¹³ | −14.9 | −20.5 | −5.3 | −6.9 | −7.8 | −3.3 | 1.9 |
| Keratin, type II cytoskeletal 1 | P04264 | 36 | 2396.7 | 0.00 | 1.8 | 3.9 | 1.5 | 6.2 | 0.0 | 2.3 | 0.0 |
| Keratin, type II cytoskeletal 2 | P35908 | 7 | 441.6 | 5.65 × 10⁻¹¹ | 2.1 | 4.3 | 3.6 | 7.1 | 2.1 | 0.0 | −2.2 |
| epidermal | | | | | | | | | | | |
| Keratin, type II cytoskeletal 2 | Q01546 | 5 | 343.6 | 3.65 × 10⁻¹¹ | 92.1 | 2.1 | 0.0 | 24.3 | 0.0 | 1.5 | −2.5 |
| oral | | | | | | | | | | | |
| Keratin, type II cytoskeletal 4 | P19013 | 33 | 1931.5 | 0.00 | 0.0 | 8.0 | 0.0 | 5.1 | 0.0 | 2.6 | 2.0 |
| Keratin, type II cytoskeletal 5 | P13647 | 23 | 1403.5 | 3.44 × 10⁻¹⁵ | 4.3 | 16.3 | 2.3 | 12.7 | 3.0 | 2.5 | 3.0 |
| Keratin, type II cytoskeletal 6A | P02538 | 63 | 3344.7 | 0.00 | 1.6 | 4.0 | 0.0 | 7.6 | 2.1 | 1.6 | 0.0 |
| Keratin, type II cytoskeletal 6B | P04259 | 2 | 125.6 | 5.61 × 10⁻¹⁴ | 3.1 | 14.7 | 2.5 | 17.0 | 4.1 | 3.5 | −1.7 |
| Keratin, type II cytoskeletal 6C | P48668 | 1 | 63.8 | 5.43 × 10⁻⁸ | −3.2 | 3.4 | 6.5 | 11.3 | 2.2 | 4.4 | 2.0 |
| Keratin, type II cytoskeletal 74 | Q7RTS7 | 1 | 24.1 | 0.00 | 5268.7 | 9742.2 | 789.6 | 13424.4 | 1580.5 | 1016.7 | 0.0 |
| Keratinocyte differentiation- | P60985 | 1 | 68.2 | 3.72 × 10⁻⁵ | −2.4 | −6.2 | −2.3 | −6.2 | 2.1 | 2.4 | 1.5 |
| associated protein | | | | | | | | | | | |
| Kininogen-1 | P01042 | 1 | 45.8 | 1.34 × 10⁻⁹ | −1.8 | −4.6 | −38.3 | −88.6 | −6.0 | 0.0 | 6.1 |
| Lactotransferrin | P02788 | 27 | 1726.8 | 0.00 | −1.8 | 0.0 | 0.0 | −5.0 | 0.0 | −2.7 | 0.0 |
| Lamin-B1 | P20700 | 3 | 103.8 | 5.79 × 10⁻⁷ | −1.8 | 1.9 | 0.0 | 2.8 | 3.2 | 0.0 | 0.0 |
| Legumain | Q99538 | 2 | 118.1 | 1.33 × 10⁻⁸ | −2.6 | 0.0 | −1.5 | 1.7 | 0.0 | 0.0 | 3.4 |
| Leucine-rich alpha-2- | P02750 | 2 | 106.7 | 1.35 × 10⁻⁹ | 0.0 | −3.4 | 0.0 | −10.9 | 0.0 | −4.0 | −6.4 |
| glycoprotein | | | | | | | | | | | |
| Leucine-rich repeat- | Q6NSJ5 | 1 | 20.1 | 3.19 × 10⁻⁶ | 3.8 | −1.5 | 1.6 | −17.2 | 0.0 | 0.0 | −205.8 |
| containing protein 8E | | | | | | | | | | | |
| Leukocyte elastase inhibitor | P30740 | 28 | 2229.4 | 4.37 × 10⁻¹⁰ | −2.0 | 0.0 | 0.0 | −1.5 | 0.0 | −2.4 | −2.5 |
| L-lactate dehydrogenase 1 | P62052 | 2 | 102.2 | 2.76 × 10⁻⁸ | −1.3 | −1.3 | −1.8 | −3.1 | 1.4 | 1.1 | 1.8 |
| OS = Lactobacillus johnsonii | | | | | | | | | | | |
| Long palate, lung and nasal | Q8TDL5 | 17 | 962.4 | 1.29 × 10⁻⁸ | 0.0 | 0.0 | −2.1 | −1.7 | −1.8 | 0.0 | 0.0 |
| epithelium carcinoma-associated | | | | | | | | | | | |
| protein 1 | | | | | | | | | | | |
| Lumican | P51884 | 1 | 30.5 | 1.24 × 10⁻⁸ | −20.2 | −9.9 | −5.2 | −21.0 | −7.4 | −19.2 | 5.0 |
| Lymphocyte-specific protein 1 | P33241 | 3 | 103.4 | 2.07 × 10⁻⁵ | 2.5 | 2.8 | 3.2 | 3.2 | 1.7 | 0.0 | −2.1 |
| Lysosome-associated | P13473 | 2 | 124.7 | 1.55 × 10⁻¹⁰ | −2.6 | 0.0 | 0.0 | −1.8 | −1.6 | −3.1 | 3.6 |
| membrane glycoprotein 2 | | | | | | | | | | | |
| Macrophage migration | P14174 | 2 | 121.5 | 9.33 × 10⁻¹⁵ | 0.0 | −4.9 | 0.0 | −9.2 | −2.8 | 1.6 | −1.6 |
| inhibitory factor | | | | | | | | | | | |
| Macrophage-capping protein | P40121 | 6 | 244.6 | 7.36 × 10⁻¹¹ | −1.7 | −5.3 | 0.0 | −3.7 | −2.1 | −2.0 | −2.4 |
| Malate dehydrogenase, | P17505 | 2 | 102.0 | 7.64 × 10⁻¹⁰ | −2.0 | −9.1 | −1.6 | −3.1 | −2.3 | −2.2 | −1.3 |
| mitochondrial OS = | | | | | | | | | | | |
| Saccharomyces cerevisiae | | | | | | | | | | | |
| Matrix metalloproteinase-9 | P14780 | 11 | 589.2 | 2.06 × 10⁻¹³ | −1.5 | −3.3 | −2.0 | −6.5 | −2.4 | −4.3 | 0.0 |
| Microtubule-associated | P27816 | 6 | 175.6 | 3.74 × 10⁻⁶ | 0.0 | 0.0 | 0.0 | 0.0 | 0.0 | −2.0 | 5.7 |
| protein 4 | | | | | | | | | | | |
| Mucin-16 | Q8WXI7 | 32 | 856.2 | 5.32 × 10⁻⁵ | 0.0 | 0.0 | 0.0 | 0.0 | 0.0 | 1.7 | 1.8 |
| Mucin-5AC (Fragments) | P98088 | 5 | 166.1 | 2.06 × 10⁻⁷ | −2.0 | −1.9 | −1.6 | −2.1 | −1.9 | −2.3 | 2.1 |
| Mucin-5B | Q9HC84 | 10 | 367.1 | 1.84 × 10⁻¹⁰ | 0.0 | −2.7 | 0.0 | −2.1 | −1.6 | 0.0 | 0.0 |
| Myeloblastin | P24158 | 13 | 694.5 | 3.33 × 10⁻¹⁶ | −2.2 | 0.0 | 1.5 | −3.1 | −3.0 | −4.0 | −1.7 |
| Myeloperoxidase | P05164 | 28 | 1652.5 | 1.49 × 10⁻¹² | −1.7 | 1.9 | 1.8 | 1.5 | 1.6 | 0.0 | 2.2 |
| N(4)-(beta-N- | P20933 | 2 | 56.8 | 2.99 × 10⁻⁹ | −1.5 | 0.0 | 0.0 | 0.0 | 0.0 | −2.0 | 5.2 |
| acetylglucosaminyl)-L- | | | | | | | | | | | |
| asparaginase | | | | | | | | | | | |
| Neuroblast differentiation- | Q09666 | 40 | 1601.4 | 3.54 × 10⁻¹¹ | −2.1 | 0.0 | 0.0 | 0.0 | −1.6 | −2.1 | −2.3 |
| associated protein AHNAK | | | | | | | | | | | |
| Neutrophil collagenase | P22894 | 9 | 519.7 | 8.86 × 10⁻¹³ | −2.4 | −4.5 | 0.0 | −5.8 | 0.0 | −1.7 | −1.6 |
| Neutrophil elastase | P08246 | 11 | 455.5 | 1.66 × 10⁻⁸ | 0.0 | 1.9 | 2.3 | 1.5 | 0.0 | 0.0 | 3.3 |
| Neutrophil gelatinase- | P80188 | 18 | 1097.5 | 1.10 × 10⁻⁷ | 0.0 | 0.0 | 0.0 | 1.5 | 0.0 | 0.0 | 0.0 |
| associated lipocalin | | | | | | | | | | | |
| Non-specific cytotoxic cell | Q6ZVX7 | 3 | 110.8 | 1.88 × 10⁻⁸ | 0.0 | 0.0 | 0.0 | 0.0 | −2.8 | 3.7 | −2.5 |
| receptor protein 1 homolog | | | | | | | | | | | |
| Nucleolar protein 8 | Q76FK4 | 3 | 85.6 | 3.79 × 10⁻⁸ | 0.0 | 0.0 | −1.9 | 2.1 | 0.0 | 0.0 | 4.2 |
| Nucleolysin TIAR | Q01085 | 1 | 82.5 | 2.66 × 10⁻⁹ | −3.6 | 0.0 | 0.0 | 0.0 | 0.0 | −4.1 | −2.3 |
| Nucleoside diphosphate | P22392 | 4 | 128.9 | 5.91 × 10⁻¹³ | 1.6 | −3.4 | 0.0 | −7.3 | −2.2 | 0.0 | −1.8 |
| kinase B | | | | | | | | | | | |
| Obscurin-like protein 1 | O75147 | 2 | 61.2 | 2.79 × 10⁻¹¹ | −1.5 | −6.1 | −2.1 | −3.3 | −1.8 | 0.0 | 0.0 |
| Olfactomedin-4 | Q6UX06 | 5 | 208.5 | 1.44 × 10⁻¹⁰ | 0.0 | −1.6 | 0.0 | 0.0 | 0.0 | 1.8 | 1.9 |
| Pantothenate synthetase OS = | Q5HL36 | 1 | 55.1 | 8.51 × 10⁻¹⁰ | −1.3 | −3.4 | −2.4 | 1.5 | 1.3 | 1.3 | 1.2 |
| Staphylococcus epidermidis | | | | | | | | | | | |
| Peptidoglycan recognition | O75594 | 1 | 42.1 | 5.35 × 10⁻⁵ | 3.9 | −2.1 | 3.1 | 1.8 | 2.6 | 0.0 | 5.4 |
| protein 1 | | | | | | | | | | | |
| Perilipin-3 | O60664 | 4 | 180.1 | 2.44 × 10⁻⁹ | 0.0 | 1.7 | 1.7 | 2.9 | 0.0 | 2.3 | 2.1 |
| Periplakin | O60437 | 7 | 315.2 | 1.45 × 10⁻¹³ | 0.0 | 3.6 | 0.0 | 3.4 | 0.0 | 2.0 | 1.8 |
| Peroxiredoxin-6 | P30041 | 3 | 151.5 | 5.60 × 10⁻⁷ | −3.0 | −2.0 | −5.8 | 0.0 | −7.6 | 0.0 | 1.5 |
| Phosphatidylethanolamine- | P30086 | 5 | 359.6 | 1.94 × 10⁻¹³ | 0.0 | −3.1 | 0.0 | −4.2 | 0.0 | −1.5 | −2.7 |
| binding protein 1 | | | | | | | | | | | |
| Phosphocarrier protein HPr | Q9KJV3 | 3 | 139.3 | 8.87 × 10⁻¹¹ | 1.1 | 1.5 | 1.1 | 2.5 | 1.2 | 1.2 | 1.2 |
| OS = Lactobacillus casei | | | | | | | | | | | |
| Phosphoglycerate kinase 1 | P00558 | 11 | 487.8 | 7.24 × 10⁻¹⁴ | 0.0 | −1.9 | 0.0 | −2.4 | 0.0 | 2.9 | 0.0 |
| Phosphoglycerate kinase | Q042F2 | 1 | 36.1 | 1.65 × 10⁻² | 4.9 | −22.1 | −1.3 | 2.5 | 1.1 | 2.1 | 3.1 |
| OS = Lactobacillus gasseri | | | | | | | | | | | |
| Phosphoglycerate kinase | A8YUE3 | 4 | 204.4 | 1.07 × 10⁻¹² | 1.4 | −1.1 | 1.9 | 2.5 | −2.5 | −1.1 | −1.1 |
| OS = Lactobacillus helveticus | | | | | | | | | | | |
| Phosphoglycerate kinase | Q9PQL2 | 1 | 29.1 | 2.25 × 10⁻¹⁰ | 7.8 | 4.4 | 1.7 | 7.1 | 1.7 | 5.1 | 7.7 |
| OS = Ureaplasma parvum | | | | | | | | | | | |
| Phospholipase B-like 1 | Q6P4A8 | 5 | 237.0 | 5.09 × 10⁻¹⁰ | −2.1 | −1.6 | 0.0 | −2.1 | −2.1 | −3.0 | 0.0 |
| Plakophilin-1 | Q13835 | 4 | 235.4 | 1.41 × 10⁻⁸ | 2.7 | 1.5 | 2.0 | 2.3 | 1.7 | −1.7 | −4.0 |
| Plasma protease C1 inhibitor | P05155 | 6 | 398.3 | 9.94 × 10⁻⁶ | 1.6 | 0.0 | −1.5 | −2.2 | −1.6 | 0.0 | 1.8 |
| Plasma serine protease inhibitor | P05154 | 10 | 606.7 | 2.63 × 10⁻⁸ | 0.0 | −1.5 | −1.9 | 0.0 | 0.0 | −1.8 | 0.0 |
| Plasminogen activator inhibitor 2 | P05120 | 5 | 233.0 | 1.03 × 10⁻¹² | −1.8 | −1.9 | −1.5 | 2.4 | −1.5 | 0.0 | 0.0 |
| Plastin-2 | P13796 | 28 | 1709.9 | 4.72 × 10⁻¹⁰ | −2.1 | −2.3 | 0.0 | −6.2 | −1.7 | −4.5 | 0.0 |
| Plastin-3 | P13797 | 9 | 475.1 | 1.21 × 10⁻¹³ | −3.7 | −6.7 | 0.0 | −5.1 | −2.0 | 0.0 | 2.0 |
| Poly(U)-specific endoribonuclease | P21128 | 1 | 63.7 | 2.42 × 10⁻⁴ | −1.8 | −30.8 | 1.5 | −4.6 | 2.0 | 1.7 | 3.1 |
| Polymeric immunoglobulin receptor | P01833 | 11 | 687.7 | 2.75 × 10⁻⁷ | 0.0 | 0.0 | 0.0 | 0.0 | 0.0 | −1.6 | 0.0 |
| POU domain, class 2, | P14859 | 5 | 128.1 | 8.44 × 10⁻⁶ | 1.9 | 3.0 | 0.0 | 2.1 | −1.5 | 0.0 | 0.0 |
| transcription factor 1 | | | | | | | | | | | |
| Probable DNA helicase II homolog | P47486 | 3 | 79.4 | 1.87 × 10⁻¹³ | −2.2 | 1.1 | −5.0 | −6.4 | −6.9 | −1.4 | −3.2 |
| OS = Mycoplasma genitalium | | | | | | | | | | | |
| Probable phosphoglycerate mutase 4 | Q8N0Y7 | 1 | 53.4 | 1.60 × 10⁻² | 7.2 | 5.6 | 1.7 | 7.0 | 1.8 | 19.2 | 29.2 |
| Profilin-1 | P07737 | 6 | 384.3 | 1.47 × 10⁻¹⁰ | 0.0 | −5.1 | −2.9 | −5.0 | −1.9 | −2.0 | −4.5 |
| Prostaglandin-H2 D- | P41222 | 2 | 108.4 | 1.61 × 10⁻⁶ | 0.0 | 1.9 | 1.6 | 0.0 | 0.0 | 0.0 | 0.0 |
| isomerase | | | | | | | | | | | |
| Prostasin | Q16651 | 3 | 128.3 | 5.06 × 10⁻⁷ | 1.7 | 0.0 | 0.0 | 0.0 | 0.0 | −1.5 | −3.2 |
| Prostate stem cell antigen | O43653 | 1 | 63.7 | 1.95 × 10⁻¹² | 14.3 | 142.1 | 3.5 | 63.8 | 25.9 | 32.9 | 11.3 |
| Prostatic acid phosphatase | P15309 | 3 | 109.3 | 2.25 × 10⁻⁹ | −3.7 | −11.1 | −1.6 | 0.0 | −1.9 | −1.5 | 3.1 |
| Proteasome subunit beta type-3 | P49720 | 2 | 63.0 | 2.49 × 10⁻⁸ | −3.6 | −5.8 | −3.1 | −5.7 | −3.5 | −5.1 | −27.7 |
| Protein disulfide-isomerase | P07237 | 10 | 445.8 | 2.05 × 10⁻⁹ | −2.3 | −1.8 | 0.0 | −5.1 | −2.9 | 0.0 | 0.0 |
| Protein FAM25 | B3EWG3 | 1 | 42.5 | 0.00 | 1874.1 | 6741.8 | 437.6 | 8944.5 | 5346.2 | 2938.9 | 0.0 |
| Protein NDRG1 | Q92597 | 1 | 38.2 | 0.00 | 2.8 | 5.4 | 3.1 | 7.0 | 6.3 | 1.8 | −1317.2 |
| Protein S100-A12 | P80511 | 6 | 406.2 | 1.76 × 10⁻⁹ | 2.3 | −1.7 | 2.6 | 0.0 | 1.7 | −2.1 | −1.5 |
| Protein S100-A7 | P31151 | 8 | 465.2 | 1.11 × 10⁻¹⁶ | −2.5 | −19.2 | 0.0 | −5.7 | −4.3 | −2.5 | −1.5 |
| Protein S100-A8 | P05109 | 16 | 712.4 | 3.33 × 10⁻¹⁶ | 0.0 | −3.1 | 0.0 | −2.5 | −2.9 | 1.5 | −1.5 |
| Protein S100-A9 | P06702 | 20 | 1133.2 | 0.00 | 0.0 | −4.2 | 0.0 | −2.6 | 0.0 | 0.0 | −3.3 |
| Protein S100-P | P25815 | 1 | 86.1 | 2.27 × 10⁻⁷ | 1.6 | 0.0 | 0.0 | 0.0 | −3.4 | 1.5 | −443.4 |
| Protein-glutamine gamma- | Q08188 | 26 | 1518.6 | 1.32 × 10⁻¹² | −1.9 | 0.0 | 0.0 | 0.0 | 0.0 | −1.6 | 0.0 |
| glutamyltransferase E | | | | | | | | | | | |
| Protein-glutamine gamma- | P22735 | 6 | 304.3 | 4.91 × 10⁻¹¹ | −2.3 | 0.0 | −1.9 | 0.0 | −2.1 | −3.4 | −5.7 |
| glutamyltransferase K | | | | | | | | | | | |
| Purine nucleoside phosphorylase | P00491 | 2 | 104.9 | 1.34 × 10⁻⁸ | 0.0 | 0.0 | −1.9 | 3.7 | −1.7 | 2.0 | 0.0 |
| Puromycin-sensitive aminopeptidase | P55786 | 6 | 240.7 | 2.54 × 10⁻¹² | −3.8 | −16.3 | −3.8 | −6.7 | −8.7 | −1.6 | 3.2 |
| Putative beta-actin-like protein 3 | Q9BYX7 | 5 | 205.9 | 5.48 × 10⁻¹¹ | 2.1 | 0.0 | 2.1 | −2.0 | 1.6 | 2.6 | 2.3 |
| Putative fatty acid-binding protein | A8MUU1 | 3 | 72.5 | 3.04 × 10⁻² | −2.0 | 1.6 | −1.6 | 1.8 | −2.0 | 0.0 | −2.6 |
| 5-like protein 3 | | | | | | | | | | | |
| Putative uncharacterized protein | P33666 | 2 | 79.6 | 2.48 × 10⁻⁸ | −2.1 | 1.0 | −1.1 | 1.9 | 2.1 | 1.0 | −2.1 |
| ydbA OS = Escherichia coli | | | | | | | | | | | |
| Pyruvate kinase isozymes M1/M2 | P14618 | 17 | 955.5 | 2.22 × 10⁻¹⁶ | 0.0 | −2.5 | −1.9 | −3.7 | −2.1 | 1.6 | −1.9 |
| Pyruvate kinase OS = | P34038 | 5 | 342.4 | 2.36 × 10⁻⁵ | −1.1 | 1.5 | −1.1 | 1.1 | −1.2 | 1.3 | −1.3 |
| Lactobacillus delbrueckii subsp. | | | | | | | | | | | |
| bulgaricus | | | | | | | | | | | |
| Pyruvate kinase OS = | Q2YTE3 | 1 | 40.6 | 6.68 × 10⁻⁸ | −1.5 | −2.6 | −3.0 | −1.1 | 1.0 | −3.4 | 1.1 |
| Staphylococcus aureus | | | | | | | | | | | |
| Ras GTPase-activating-like | P46940 | 4 | 139.2 | 7.55 × 10⁻¹⁵ | −1.9 | −3.9 | −2.1 | −12.8 | −2.5 | −2.7 | 0.0 |
| protein IQGAP1 | | | | | | | | | | | |
| Ras-related protein Rab-7a | P51149 | 5 | 311.8 | 8.85 × 10⁻⁹ | −1.6 | 0.0 | 1.5 | −1.6 | 0.0 | −2.5 | −1.5 |
| Repetin | Q6XPR3 | 7 | 192.1 | 2.22 × 10⁻¹⁶ | 9.6 | 4.3 | 1.5 | 13.4 | 1.9 | 3.6 | 1.8 |
| Rho GDP-dissociation inhibitor 2 | P52566 | 2 | 76.2 | 1.67 × 10⁻¹¹ | 0.0 | −3.4 | −3.2 | −5.2 | −1.7 | −3.3 | 0.0 |
| Ribosome-recycling factor OS = | B7GQS1 | 2 | 71.2 | 1.25 × 10⁻¹⁴ | −1.2 | −4.5 | −1.1 | −2.4 | 1.0 | −2.8 | −3.4 |
| Bifidobacterium longum subsp. | | | | | | | | | | | |
| infantis | | | | | | | | | | | |
| Sciellin | O95171 | 7 | 366.1 | 1.26 × 10⁻⁷ | 0.0 | 0.0 | 0.0 | 0.0 | 0.0 | 0.0 | 2.0 |
| Semenogelin-2 | Q02383 | 2 | 70.7 | 2.74 × 10⁻⁵ | 525.8 | 1.9 | −2.3 | 5.9 | −1.8 | 0.0 | 4.9 |
| Serine protease 27 | Q9BQR3 | 4 | 190.7 | 2.61 × 10⁻¹³ | 0.0 | −1.7 | 0.0 | −2.3 | 0.0 | −1.5 | −1.5 |
| Serine/threonine-protein kinase | O75385 | 3 | 94.6 | 2.53 × 10⁻¹³ | −5.1 | −2.2 | 0.0 | −4.4 | −1.7 | −7.4 | −5.7 |
| ULK1 | | | | | | | | | | | |
| Serotransferrin | P02787 | 35 | 1958.4 | 2.78 × 10⁻¹⁵ | 1.6 | −1.5 | 0.0 | −3.9 | −1.5 | 0.0 | 0.0 |
| Serpin B12 | Q96P63 | 7 | 448.5 | 2.25 × 10⁻⁸ | −1.5 | −2.7 | −1.8 | −1.9 | 0.0 | −1.7 | −2.7 |
| Serpin B13 | Q9UIV8 | 18 | 1048.5 | 6.91 × 10⁻⁵ | 0.0 | 0.0 | 1.5 | 0.0 | 0.0 | 0.0 | 0.0 |
| Serpin B3 | P29508 | 60 | 3860.2 | 0.00 | 0.0 | 3.2 | 1.8 | 4.3 | 1.6 | 2.6 | 0.0 |
| Serpin B4 | P48594 | 10 | 814.8 | 3.10 × 10⁻⁵ | 0.0 | 0.0 | 1.5 | 0.0 | 0.0 | 0.0 | 0.0 |
| Serpin B5 | P36952 | 5 | 209.5 | 2.56 × 10⁻¹⁴ | −3.3 | −7.9 | 0.0 | −4.7 | −2.6 | 2.0 | 2.1 |
| Serpin B6 | P35237 | 8 | 422.3 | 5.02 × 10⁻¹⁰ | −2.1 | 0.0 | 0.0 | 0.0 | 0.0 | −2.2 | −2.3 |
| Serpin B9 | P50453 | 2 | 76.8 | 2.52 × 10⁻¹² | −2.6 | −4.1 | 0.0 | −5.6 | 0.0 | −6.6 | 2.7 |
| Serum albumin | P02768 | 89 | 4758.9 | 1.55 × 10⁻¹² | −1.5 | −2.3 | 0.0 | −13.2 | −1.6 | −2.1 | −2.7 |
| Serum paraoxonase/arylesterase 1 | P27169 | 5 | 174.3 | 2.28 × 10⁻⁷ | 1.5 | 0.0 | 1.7 | 0.0 | 1.5 | 0.0 | −4.3 |
| Small proline-rich protein 3 | Q9UBC9 | 28 | 1125.2 | 0.00 | 3.4 | 6.6 | 0.0 | 6.6 | 1.7 | 4.4 | 0.0 |
| Stanniocalcin-1 | P52823 | 1 | 48.3 | 5.28 × 10⁻⁵ | 1.9 | 3.4 | 1.7 | 113.1 | 4.0 | 13.3 | −1.7 |
| Sulfhydryl oxidase 1 | O00391 | 13 | 586.5 | 9.15 × 10⁻¹¹ | −1.6 | 0.0 | 0.0 | 1.6 | 0.0 | −1.6 | 1.7 |
| Superoxide dismutase | P00441 | 5 | 189.0 | 1.94 × 10⁻⁶ | 0.0 | −1.8 | 0.0 | −1.5 | 0.0 | 0.0 | 0.0 |
| [Cu—Zn] | | | | | | | | | | | |
| Suprabasin | Q6UWP8 | 4 | 290.5 | 1.91 × 10⁻⁴ | 13.5 | 17.9 | 7.6 | 15.1 | 3.9 | 14.4 | −11.5 |
| Syntenin-1 | O00560 | 1 | 65.9 | 9.80 × 10⁻³ | 4.4 | 1.9 | 2.6 | −3.1 | 0.0 | −2.3 | −5.8 |
| Talin-1 | Q9Y490 | 12 | 403.0 | 8.18 × 10⁻⁸ | 0.0 | −1.6 | 0.0 | −1.6 | 0.0 | 0.0 | 0.0 |
| Tctex1 domain-containing | Q8WW35 | 1 | 45.1 | 1.64 × 10⁻⁸ | −1.6 | 1.8 | −25.1 | 4.2 | −13.6 | 0.0 | −6.4 |
| protein 2 | | | | | | | | | | | |
| Teneurin-3 | Q9P273 | 1 | 47.0 | 4.91 × 10⁻¹² | 9.7 | 33.3 | 12.3 | −2.3 | 10.2 | 0.0 | 0.0 |
| Thioredoxin | P10599 | 9 | 485.1 | 2.83 × 10⁻¹¹ | 0.0 | 2.5 | 1.5 | 3.1 | 0.0 | 0.0 | 0.0 |
| Transaldolase | P37837 | 6 | 273.6 | 2.45 × 10⁻¹³ | −1.8 | −1.9 | 1.6 | −1.7 | −2.2 | 2.4 | 0.0 |
| Transmembrane protease | O60235 | 9 | 566.0 | 1.13 × 10⁻¹¹ | −1.8 | 0.0 | −1.6 | 2.6 | 0.0 | 0.0 | 0.0 |
| serine 11D | | | | | | | | | | | |
| Transthyretin | P02766 | 8 | 576.3 | 3.77 × 10⁻⁹ | 0.0 | 0.0 | 0.0 | −4.7 | −1.8 | 0.0 | 0.0 |
| Trigger factor OS = | Q03QN6 | 1 | 34.9 | 6.44 × 10⁻¹¹ | −2.4 | −3.8 | −2.1 | −2.2 | −3.2 | −2.7 | −1.1 |
| Lactobacillus brevis | | | | | | | | | | | |
| Triosephosphate isomerase | Q1GB25 | 4 | 149.1 | 1.50 × 10⁻⁸ | −1.2 | −1.3 | 2.4 | −1.2 | −1.3 | −1.4 | 1.0 |
| OS = Lactobacillus delbrueckii | | | | | | | | | | | |
| subsp. bulgaricus | | | | | | | | | | | |
| Tumor protein D54 | O43399 | 2 | 98.8 | 1.67 × 10⁻¹⁰ | 0.0 | 2.2 | 0.0 | 2.2 | 0.0 | 0.0 | 4.1 |
| Uncharacterized membrane | P31379 | 2 | 80.8 | 5.18 × 10⁻¹⁴ | 2.0 | 1.1 | 2.0 | 2.9 | 4.2 | −1.6 | 2.6 |
| protein YAL018C OS = | | | | | | | | | | | |
| Saccharomyces cerevisiae | | | | | | | | | | | |
| Uncharacterized protein yphG | P76585 | 1 | 47.3 | 7.63 × 10⁻⁵ | −4.3 | 1.1 | −1.1 | 1.1 | −1.1 | 1.0 | −1.7 |
| OS = Escherichia coli | | | | | | | | | | | |
| UPF0082 protein SAB0618 | Q2YSR2 | 1 | 40.4 | 2.67 × 10⁻⁸ | 5.6 | 9.3 | 9.2 | 10.5 | 5.9 | 8.9 | 1.8 |
| OS = Staphylococcus aureus | | | | | | | | | | | |
| Uronyl 2-sulfotransferase | Q9Y2C2 | 1 | 27.4 | 1.03 × 10⁻⁴ | −786.0 | 2.0 | −786.0 | 2.1 | −917.5 | −7.7 | 4.3 |
| UTP--glucose-1-phosphate | Q16851 | 2 | 76.2 | 1.04 × 10⁻⁸ | 0.0 | 0.0 | 0.0 | 0.0 | 0.0 | 0.0 | 4.9 |
| uridylyltransferase | | | | | | | | | | | |
| UV excision repair protein | P54727 | 5 | 181.1 | 3.03 × 10⁻¹⁰ | −2.9 | −2.8 | −1.6 | −2.2 | −1.5 | −2.2 | 3.6 |
| RAD23 homolog B | | | | | | | | | | | |
| Versican core protein | P13611 | 7 | 299.5 | 6.15 × 10⁻⁸ | 0.0 | 0.0 | −1.7 | 2.6 | 0.0 | 0.0 | 2.4 |
| Vinculin | P18206 | 17 | 850.8 | 3.14 × 10⁻⁹ | 0.0 | 0.0 | 1.5 | 0.0 | 0.0 | 0.0 | 0.0 |
| Vitamin D-binding protein | P02774 | 3 | 112.5 | 6.66 × 10⁻¹⁶ | 0.0 | −1.7 | −2.0 | −17.8 | 0.0 | 0.0 | −3.4 |
| Werner syndrome ATP- | Q14191 | 1 | 32.6 | 4.80 × 10⁻¹³ | 302.6 | 331.1 | 131.2 | 750.5 | 12.4 | 209.6 | 5.7 |
| dependent helicase | | | | | | | | | | | |
| Zinc finger protein 185 | O15231 | 5 | 171.4 | 3.62 × 10⁻¹¹ | 2.2 | 5.2 | 0.0 | 5.8 | 1.5 | 1.8 | 5.3 |
| Zinc finger protein 335 | Q9H4Z2 | 4 | 122.6 | 1.90 × 10⁻⁷ | 0.0 | 0.0 | 0.0 | −1.6 | 0.0 | 0.0 | −2.1 |
| Zinc-alpha-2-glycoprotein | P25311 | 2 | 62.2 | 1.98 × 10⁻¹⁰ | 0.0 | 0.0 | 0.0 | −9.4 | 0.0 | 0.0 | −1.8 |
| Zyxin | Q15942 | 5 | 138.5 | 5.10 × 10⁻¹² | −1.7 | −3.2 | 0.0 | −5.1 | 2.0 | 0.0 | 0.0 |

Proteins identified by proteomic techniques were submitted for Gene Ontology (GO) analysis (AmiGO version 1.8, database release Mar. 11, 2012) to identify biological processes, molecular functions and subcellular localizations associated with the identified proteins. MS/MS data were further evaluated for tissue expression patterns using the publicly available Human Protein Atlas database (HPA). Table 9 reports the Gene Ontology (GO) categorization of the MS/MS identified proteins differently expressed in healthy and BV affected women. Classification was performed according to “biological process” as keyword category.

TABLE 9

| | Percentage (%) of GO |
|---|---|
| | categorization by “biological |
| Gene Ontology (GO) categorization | process” |
| Acute-phase and inflammatory response | 5 |
| Blood coagulation | 5 |
| Cell adhesion | 3 |
| Cytoskeleton organization | 2 |
| Epidemis development and keritinisation | 13 |
| Innate immune response and complement | 20 |
| activation | |
| Regulation of endopeptidase activity | 4 |
| Response to oxidative stress and external | 5 |
| stimulus | |
| Small molecule metabolic process | 12 |
| Transport | 11 |
| Others | 7 |
| NA | 13 |

Table 10 reports the Gene Ontology (GO) categorization of the MS/MS identified proteins differently expressed in healthy and BV affected women. Classification was performed according to “cellular component” as keyword category.

TABLE 10

| | Percentage (%) of GO |
|---|---|
| | categorization by “cellular |
| Gene Ontology (GO) categorization | component” |
| Cytoplasm | 22 |
| Cytoskeleton constituent | 3 |
| Extracellular space | 35 |
| Granules and vesicles | 2 |
| Haptoglobin-hemoglobin complex | 2 |
| Integral or associated to plasma membrane | 15 |
| Internal organelle | 8 |
| Keratin filament | 5 |
| Nucleus | 2 |
| NA | 6 |

Table 11 reports the Gene Ontology (GO) categorization of the MS/MS identified proteins differently expressed in healthy and BV affected women. Classification was performed according to “molecular function” as keyword category.

TABLE 11

| Gene | Percentage (%) of GO categorization |
|---|---|
| Ontology (GO) categorization | by “molecular function” |
| Antigen binding | 12 |
| Calcium ion binding | 13 |
| Ion binding | 2 |
| Lipid binding | 2 |
| Protein binding | 19 |
| Enzymatic activity | 19 |
| Receptor activity | 2 |
| Structural molecule activity | 16 |
| Transporter activity | 5 |
| Others | 2 |
| NA | 8 |

A multivariate analysis (PCA) was done with the MS/MS data of differently expressed proteins and represented in FIG. 2. FIG. 2a represents the PCA of the peptides in the fractionated pool of healthy women (H) and women affected by BV at visit V1. FIG. 2b represents the PCA of the peptides in the whole pools of the women affected by BV before and after treatment with rifaximin, respectively at the dosage of 100 mg for 5 days in remission (A-R) and no remission (A-N); 25 mg for 5 days in remission (B-R) and no remission (B-N); 100 mg for 2 days in remission (C-R) and no remission (C-N) and placebo for 5 days (D-N). The date derive by the analysis if the sample in triplicate.

1. A method of diagnosing a vaginal bacterial infection in an individual undergoing testing for said infection, the method comprising:
subjecting to proteomic analysis a test vaginal fluid sample obtained from the individual undergoing testing;
detecting expression levels of one or more proteins in the test vaginal fluid sample; and
reporting results of the detecting to provide treatment for the vaginal bacterial infection to the individual undergoing testing based on a detected decrease or increase of expression levels of the one or more proteins in the test vaginal fluid sample compared with expression levels of the one or more proteins in a reference sample representing vaginal fluid from a healthy or uninfected individual.

2. The method of claim 1, wherein the one or more proteins are selected from the group consisting of Vitamin D binding protein, Desmocollin-2, Calcium-activated chloride channel regulator 4, Catalase, Small proline-rich protein 3, Galectin-3-binding protein, Hemopexin, Immunoglobulin family, Intermediate filament family, Lipocalin family, Alpha 1-acid glycoprotein 1, Alpha-1-acid glycoprotein 2, Neutrophil gelatinase-associated lipocalin, Limphocyte-specific protein 1, Myeloblastin, Perilipin-3, Perilplakin, Protein S100-A9, Protein S100-A7, and Superoxide dismutase [Cu—Zn].

3. The method of claim 1, wherein the one or more proteins are selected from the group consisting of Desmocollin-2, Small proline-rich protein 3, Immunoglobulin J chain, keratin type I cytoskeletal 10, keratin type II cytoskeletal 1, keratin type II cytoskeletal 2 epidermal, keratin type II cytoskeletal 5, Neutrophil gelatinase-associated lipocalin, Limphocyte-specific protein 1, Perilipin-3, and Perilplakin and the treatment is provided based on a detected increase in the expression levels of the one or more proteins.

4. The method of claim 1, wherein the one or more proteins are selected from the group consisting of Vitamin D binding protein, Calcium-activated chloride channel regulator 4, Catalase, Galectin-3-binding protein, Hemopexin, IgM chain constant region, alpha-1-acid glycoprotein 1, alpha-1-acid glycoprotein 2, Myeloblastin, Protein S100-A9, Protein S100-A7, and Superoxide dismutase [Cu—Zn] and the treatment is provided based on a detected decrease in the expression levels of the one or more proteins.

5. The method of claim 1, wherein the detected increase is a ratio between the expression levels of the one or more proteins in the test vaginal fluid sample and the expression levels of the one or more proteins in the reference sample in a range from about 1.5 to about 40.

6. The method of claim 1, wherein the detected decrease is a ratio between the expression levels of the one or more proteins in the test vaginal fluid sample and the expression levels of the one or more proteins in the reference sample in a range from about −1.5 to about −5650.

7. A method of diagnosing a status of remission from a bacterial vaginal infection of an individual undergoing testing for remission after antibiotic treatment, the method comprising:
subjecting to proteomic analysis a test vaginal fluid sample obtained from the individual undergoing testing for remission after antibiotic treatment;
detecting expression levels of at least one protein in the test vaginal fluid sample; and
reporting results of the detecting to provide treatment to the individual undergoing testing for remission based on a detected decrease or increase of expression levels of the at least one protein in the test vaginal fluid sample compared to expression levels of the at least one protein in a vaginal fluid sample from an individual having the bacterial vaginal infection.

8. The method of claim 7, wherein the at least one protein is selected from the group consisting of Vitamin D binding protein, Calcium-activated chloride channel regulator 4, Catalase, Galectin-3-binding protein, Hemopexin, ImmunoglobulinM chain C region, Alpha 1-acid glycoprotein 1, Alpha-1-acid glycoprotein 2, Protein S100-A9, Protein S100-A7, and Superoxide dismutase [Cu—Zn] and the treatment is provided based on a detected decrease in the expression levels of the at least one protein.

9. The method of claim 7, wherein the at least one protein is selected from the group consisting of Desmocollin-2, Small proline-rich protein 3, Immunoglobulin J chain, Keratin, type I cytoskeletal 10, Keratin, type II cytoskeletal 1, Keratin, type II cytoskeletal 2 epidermal, Keratin, type II cytoskeletal 5, Neutrophil gelatinase-associated lipocalin, Lymphocyte-specific protein 1, Perilipin-3, and Periplakin and the treatment is provided based on a detected increase in the expression levels of the at least one protein.

10. The method of claim 7, wherein the antibiotic is rifaximin.

11. The method of claim 7, further comprising selecting an optimal or efficacious dose of antibiotic and a time of treatment on the basis of the detected decrease or increase of expression levels of the at least one protein.

12. The method of claim 7, wherein the individual undergoing testing form remission after antibiotic treatment is a patient not responding to the antibiotic treatment and wherein a status of remission from the bacterial vaginal infection after the antibiotic treatment is not identified.

13. The method according to claim 1 wherein the vaginal fluid sample is a sample of cervicovaginal fluid.

14. A test kit for diagnosing a vaginal bacterial infection, the kit comprising at least one protein useful for identifying the vaginal infection and instructions for carrying out the method of diagnosing vaginal bacterial infection according to claim 1 using mass spectrometry.

15. A test kit for determining the status of remission from a bacterial vaginal infection, the kit comprising at least one protein useful for determining the remission from vaginal infections after treatment with antibiotic, and instructions for carrying out the method of diagnosing a status of remission from a bacterial vaginal infection according to claim 7 using analytical techniques for protein determination.

16. A method for diagnosis of a vaginal infection in an individual, the method comprising
detecting proteomic profiles of proteins expressed in a test sample of a vaginal fluid obtained from the individual; and
reporting results of the detecting to provide treatment of the vaginal infection in the individual based on a detected presence of 3 or more total number of proteins differentially expressed in the detected proteomic profile of the test sample compared with proteomic profiles of the proteins expressed in a normal or a reference sample of vaginal fluid.

17. The method according to claim 16, wherein a differentially expressed protein is identified as having an absolute value of a ratio between the expression levels in detected proteomic profiles of the test sample and the expression levels of the proteomic profiles of the proteins expressed in the normal or the reference sample, equal to or greater than 1.5.

18. The method according to claim 16, wherein a differentially expressed protein is identified as having an absolute value of a ratio between the expression levels in detected proteomic profiles of the test sample and the expression levels of the proteomic profiles of the proteins expressed in the normal or the reference sample, equal to or greater than 3.

19. A method for evaluation of efficacy of treatment of vaginal infections in an individual, the method comprising
detecting proteomic profiles of a test sample of a vaginal fluid obtained from the individual during or after a course of therapy; and
reporting results of the detecting to provide treatment to the individual based on a detected presence of one or more differentially expressed proteins in the detected proteomic profiles of the tests sample compared with proteomic profiles of a sample of vaginal fluid taken before a course of therapy or at an earlier point during the course of therapy.

20. The method according to claim 19, wherein the course of therapy is administering rifaximin.

21. The method according to claim 20, wherein the rifaximin is administered in an amount between 25 mg and 100 once daily for 5 days.

22. The method according to claim 19, wherein a differentially expressed protein is identified as having an absolute value of a ratio between the expression levels in detected proteomic profiles of the test sample and the expression levels of the proteomic profiles of the sample of vaginal fluid taken before a course of therapy or at an earlier point during the course of therapy equal to or greater than 1.5.

23. The method according to claim 19, wherein a differentially expressed protein is identified as having an absolute value of a ratio between the expression levels in detected proteomic profiles of the test sample and the expression levels of the proteomic profiles of the sample of vaginal fluid taken before a course of therapy or at an earlier point during the course of therapy equal to or greater than 3.

24. The method according to claim 19, wherein if non responder status is determined, the course of therapy is modified by changing an administered antibiotic, by changing a dose size, or by changing a dosing frequency.

25. A method for identifying an efficacious treatment of vaginal infections, the method comprising:
identifying a population of individuals diagnosed with a bacterial vaginal infection,
subjecting to a pre-treatment proteomic analysis a vaginal fluid sample obtained from each individual to detect expressed proteins in the vaginal fluid of each individual before treatment;
separating the population into two or more pools of individuals;
administering a distinct therapy to each pool of individuals of the two or more pools of individuals;
subjecting to a post-treatment proteomic analysis a vaginal fluid sample obtained from each individual in each pool of individuals to proteomic analysis after administering the distinct therapy, to detect expressed proteins in the vaginal fluid of individuals in each pool of individuals after treatment;
and
reporting results of the pre-treatment proteomic analysis and the post-treatment proteomic analysis to provide treatment to an individual having a bacterial vaginal infection based on the distinct therapy administered to a pool of individuals with a greatest number of detected expressed proteins differentially expressed in said pool of individuals before and after treatment.

26. The method according to claim 25, wherein a differentially expressed protein is identified as having an absolute value of a ratio between expressed protein detected in the individuals after treatment and expressed proteins detected in the individuals before treatment equal to or greater than 1.5.

27. A method for predicting remission of a vaginal bacterial infection following treatment in an individual undergoing testing for such infection, the method comprising:
subjecting a vaginal fluid sample obtained from the individual to proteomic analysis to detect expressed proteins in the vaginal fluid sample of the individual; and
determining remission based on detected expressed proteins in the vaginal fluid sample having altered expression levels of the detected expressed proteins compared with expression levels of the detected expressed proteins in a reference sample representing vaginal fluid from a healthy or uninfected individual,
wherein the detected expressed proteins are one or more proteins selected from Table 1 and/or Table 2 and the detected expressed proteins have an absolute value of a ratio between the expression levels of the detected expressed protein in vaginal fluid sample and the expression levels of the detected expressed proteins the reference sample equal to or greater than 1.

28. The method according to claim 27, wherein the treatment is performed by administering a pharmaceutical formulation comprising rifaximin to the individual undergoing testing.

29. A method of treating a vaginal bacterial infection in an individual wherein a diagnosis of infection has been determined by the method according to claim 1, the method comprising:
administering a pharmaceutical composition comprising rifaximin to the individual in an therapeutically effective amount based on the detected decrease or increase of expression levels of the one or more proteins in the vaginal fluid sample of the individual compared with expression levels of the one or more proteins in a reference sample representing vaginal fluid from a healthy or uninfected individual.

30. The method according to claim 7, wherein the vaginal fluid sample is a sample of cervicovaginal fluid.

31. The test kit according to claim 15, wherein the antibiotic is rifaximin.
